(19) United States
(12) Patent Application Publication
(10) Pub. No.: US 2013/0059787 A1
(43) Pub. Date: Mar. 7, 2013

(54) METHODS OF USING HUMAN PROTEIN KINASE C DELTA VIII AS A BIOMARKER

(71) Applicants: Niketa A. Patel, Land O' Lakes, FL (US); Denise R. Cooper, St. Petersburg, FL (US)

(72) Inventors: Niketa A. Patel, Land O' Lakes, FL (US); Denise R. Cooper, St. Petersburg, FL (US)

(73) Assignees: UNITED STATES DEPARTMENT OF VETERAN AFFAIRS, Washington, DC (US); UNIVERSITY OF SOUTH FLORIDA, Tampa, FL (US)

(21) Appl. No.: 13/588,345

(22) Filed: Aug. 17, 2012

Related U.S. Application Data
(63) Continuation of application No. PCT/US11/25269, filed on Feb. 17, 2011.
(60) Provisional application No. 61/305,375, filed on Feb. 17, 2010.

(51) Int. Cl. A61K 38/17; A61P 25/28; C12N 5/071; C12Q 1/68; G01N 33/573; A61K 31/203; A61P 35/00

(52) U.S. Cl. 514/17.7; 514/559; 435/6.12; 435/7.4; 435/375

(57) ABSTRACT

RA treatment can improve cognition; promote neurogenesis; and regulate alternative splicing of genes, particularly by mediating mechanisms of 5′ splice site selection and generation of PKCδ alternatively spliced variants. Expression of PKCδVIII is an indicator of the levels of on-going apoptosis in neurons. In the aging brain, switching the isoform expression to PKCδVIII by RA could shield the cells from neuronal death. The inventors discovered that human PKCδVIII expression is increased in neuronal cancer and decreased in Alzheimer's disease. The data shows that PKCδVIII promotes neuronal survival and increases neurogenesis via Bcl2 and Bcl-xL. In addition, the trans-factor SC35 was found to be crucial in mediating the effects of RA on alternative splicing of PKCδVIII mRNA in neurons. The data described herein indicate that PKCδVIII can be used as a biomarker for neurological diseases such as cancers and Alzheimer's disease and as a tool for monitoring and evaluating treatment.

### CROSS REFERENCE TO RELATED APPLICATIONS

This application is a continuation of and claims priority to International Patent Application No. PCT/US11/25269 entitled “Methods of Using Human Protein Kinase C Delta VIII as a Biomarker,” filed Feb. 17, 2011, which is a non-provisional of and claims priority to U.S. Provisional Patent Application No. 61/305,375 entitled “Methods of Predicting Neurodegenerative Diseases and Neuronal Cancers Using Human Protein Kinase C Delta VIII”, filed on Feb. 17, 2010, the contents of which are herein incorporated by reference.

### GOVERNMENTAL SUPPORT

This invention was made with governmental support under Grant No. 054393 awarded by the National Institutes of Health (NIH). The government has certain rights in the invention.

### FIELD OF INVENTION

This invention relates to assays. Specifically, the invention provides a method of predicting neurodegenerative disease or neuronal cancers using biomarkers as well as a method of modulating neuronal survival; a method of modulating apoptosis; and a method of modulating PKCδ isozyme expression in cells.

### BACKGROUND OF THE INVENTION

Vitamin A:

Vitamin A is a micronutrient essential in a variety of biological actions ranging from embryogenesis, immunity, reproduction as well as in the development, regeneration and maintenance of the nervous system. Vitamin A and its metabolites regulate gene expression and play a role in the mature brain by influencing synaptic plasticity and memory and learning capabilities. The physiologically active forms of Vitamin A (VA) are: retinaldehyde (integral to phototransduction) and retinoic acid—which mediates most effects of vitamin A including, but not limited to, cellular development, differentiation, proliferation, apoptosis and regulation of gene expression. All-trans retinoic acid (RA), a mediator of vitamin A activity, is specifically involved in the developing and mature CNS as well as in the adult brain to maintain synaptic plasticity in the hippocampus which is crucial for memory and cognition. RA increases hippocampal neurogenesis and rescues most neuronal defects caused by vitamin A deficiency. (Etchamendy, N., et al., Alleviation of a selective age-related relational memory deficit in mice by pharmacologically induced normalization of brain retinoid signaling. J Neurosci, 2001. 21(16): p. 6423-9; Mingaud, F., et al., Retinoid hyposignaling contributes to aging-related decline in hippocampal function in short-term/working memory organization and long-term declarative memory encoding in mice. J Neurosci, 2008. 28(1): p. 279-291)

Vitamin A and its carotene precursors are found in a variety of foods such as red meat, liver, milk, cheese as well as in high amounts in brightly colored fruits and vegetables such as carrots, peas, beans, peaches etc. Vitamin A is stored and metabolized in the liver. The availability of VA in pre-formed sources is greater than that of precursor carotenoids. RA can traverse cell membranes and rapidly enter cells. More than 88% of RA present in the brain is derived from circulation.

Deficiency of VA results in birth defects, vision impairments and memory deficits. Vitamin A deficiency also impairs normal immune system maturation. Subjects with VA deficiency display lower antibody responses which can be enhanced by VA and RA treatment (Ross, A. C., Vitamin A supplementation and retinoic acid treatment in the regulation of antibody responses in vivo. Vitam Horm, 2007. 75: p. 197-222; Ross, A. C., Q. Chen, and Y. Ma, Augmentation of antibody responses by retinoic acid and costimulatory molecules. Semin Immunol, 2009. 21(1): p. 42-50)

On the other hand, high doses of vitamin A can result in hypervitaminosis A and induce severe developmental abnormalities and retinoid toxicity whose symptoms include alopecia, skin erythema, conjunctivitis, liver cirrhosis, peripheral neuritis etc. (Hathcock, J. N., et al., Evaluation of vitamin A toxicity. Am J Clin Nutr, 1990. 52(2): p. 183-202)

RA in the Nervous System:

Vitamin A metabolite, RA, influences a broad range of physiological and pathological processes both in embryonic CNS as well as in the mature brain. RA is a developmental molecule and promotes neuronal differentiation in the developing embryo. RA also plays a role in adult neuronal function, plasticity as well as in memory. High levels of RA are seen during development and experimentally induced deficiencies lead to several abnormalities in the development of CNS and results in impairment of hippocampal neurogenesis and spatial memory deficit. (Bonnet, E., et al., Retinoic acid restores adult hippocampal neurogenesis and reverses spatial memory deficit in vitamin A deprived rats. PLoS ONE, 2008. 3(10): p. e3487)

RA plays a role in adult brain plasticity by regulating gene expression through its nuclear receptors. Neurogenesis in the adult brain came into the limelight in the early 1990s. The birth of new neurons, outgrowth of neurites and formation of synapses are documented in the adult CNS. RA regulates the neural development, as well as its plasticity, and promotes neurogenesis. (McCaffery, P., J. Zhang, and J. E. Crandall, Retinoic acid signaling and function in the adult hippocampus. J. NeuroBiol, 2006. 66: p. 780-791)

The hippocampus is the seat of memory and learning. Neurogenesis in the adult hippocampus occurs in the subgranular zone (SGZ) at the border between the granule cell layer (GCL) and hilus of the dentate gyms. RA promotes in vitro neurogenesis and has been suggested as a therapeutic molecule to increase adult hippocampal neurogenesis. (Jacobs, S., et al., Retinoic acid is required early during adult neurogenesis in the dendate gyms. Proc Natl Acad Sci USA, 2006. 103(10): p. 3902-3907; Takahashi, J., T. D. Palmer, and F. H. Gage, Retinoic acid and neutrophins collaborate to regulate neurogenesis in adult-derived neural stem-cell cultures. J. NeuroBiol, 1999. 38(1): p. 65-81; Wang, T. W., H. Zhang, and J. M. Parent, Retinoic acid regulates postnatal neurogenesis in the murine subventricular zone-olfactory bulb pathway. Development, 2005. 132(12): p. 2721-2732) Further, RA induces dendritic growth and spine formation in the hippocampus via RARα. (Chen, N. and J. L. Napoli, All-trans-retinoic acid stimulates translation and induces spine formation in hippocampal neurons through a membrane-associated RARalpha. Faseb J, 2008. 22(1): p. 236-45)

Several in vivo studies have demonstrated that age-related neuron loss, decline in cognitive function, memory loss and onset of neurodegenerative diseases can be reversed by administration of RA. (Misner, D., et al., Vitamin A deprivation results in reversible loss of hippocampal long-term synaptic plasticity. Proc Natl Acad Sci USA, 2001. 98(20): p. 11714-11719; Enderlin, V., et al., Age-related decrease in mRNA for nuclear receptors and target genes are reversed by retinoic acid treatment. Neurosci Lett, 1997. 229(2): p. 125-129; Maden, M., Retinoic acid in the development and maintenance of the nervous system. Nature Reviews Neuroscience, 2007. 8(10): p. 755-765) RA promotes neurogenesis and survival of the neurons. RA is established as an early signaling component of the CNS and as a master switch of gene expression.

RA in Neurodegenerative Diseases:

Vitamin A and its metabolite RA have been shown to perform neuroprotective roles. Retinoid hyposignaling and activation of target gene transcription through its nuclear receptors contributes to aging-related decline in hippocampal function. (Mingaud, F., et al., Retinoid hyposignaling contributes to aging-related decline in hippocampal function in short-term/working memory organization and long-term declarative memory encoding in mice. J Neurosci, 2008. 28(1): p. 279-291) This decline in hippocampal function can be reversed by a nutritional vitamin A supplement.

There is significant evidence about the genetic linkage of RA and its receptors to Alzheimer's disease (AD). (Goodman, A. B. and A. B. Pardee, Evidence for defective retinoid transport and function in late onset Alzheimer's disease. PNAS, 2003. 100(5): p. 2901-2905) It has been demonstrated that chromosomes 10q23 and 12q13 are most frequently associated with AD. At each of these loci, genes related to retinoids have been found. Studies in Alzheimer's disease have revealed that RA signaling pathway is impaired in the brain. (Husson, m., et al., Retinoic acid normalizes nuclear receptor mediated hypo-expression of proteins involved in beta-amyloid deposits in cerebral cortex of vit A deprived rats. Neurobiol Dis, 2006. 23(1): p. 1-10) RA and its nuclear receptors regulate a number of genes that are essential in the regulation of APP processing and thus Aβ deposits. Late onset Alzheimer's disease is directly related with the availability of RA to the adult brain. (Goodman, A. B. and A. B. Pardee, Evidence for defective retinoid transport and function in late onset Alzheimer's disease. PNAS, 2003. 100(5): p. 2901-2905) A recent publication has demonstrated that RA treatment given to the Alzheimer's mouse model-APP/PS1 transgenic mice was effective in the prevention and treatment of AD. Specifically, it was shown that RA treatment: (i) decreased Aβ deposition; (ii) decreased tau phosphorylation; (iii) decreased APP phosphorylation and processing; (iv) decreased activation of microglia and astrocytes; (v) attenuated neuronal degeneration; (vi) improved spatial learning and memory. (Ding, Y., et al., Retinoic acid attenuates beta-amyloid deposition and rescues memory deficits in an Alzheimer's disease transgenic mouse model. J Neurosci, 2008. 28(45): p. 11622-34)

An ischemic stroke, caused by restricted blood flow to the brain, elicits multiple cellular processes that lead to cell death via apoptosis. Recently it has been shown that RA injections immediately and following ischemia reduced the infarct volume. Vitamin A and its derivatives are proposed as acute neuroprotective strategy for stroke. (Sato, Y., et al., Stereo-selective neuroprotection against stroke with vitamin A derivatives. Brain Res, 2008. 1241: p. 188-92; Shen, H., et al., 9-Cis-retinoic acid reduces ischemic brain injury in rodents via bone morphogenetic protein. J Neurosci Res, 2008. 87(2): p. 545-555; Li, L., et al., The effects of retinoic acid on the expression of neurogranin after experimental cerebral ischemia. Brain Res, 2008. 1226: p. 234-40)

Thus, RA is an established signaling molecule that is crucial in the development, differentiation and maintenance of the nervous system. RA promotes adult hippocampal neurogenesis and enhances survival of neurons. There are a number of excellent reviews on the neurobiology of RA signaling and its functions in neural plasticity and neurogenesis in the hippocampus; its role in disorders such as Parkinson's disease, Huntington's disease, Alzheimer's disease, and motoneuron disease as well as its effects on memory, cognition. RA acts as a transcriptional activator for numerous downstream regulatory molecules. However, the targets of RA in the brain and mechanisms underlying RA-mediated increased neuronal survival are poorly understood.

Protein Kinase C(PKC):

Activation of PKC, a serine/threonine kinase, is essential for learning, synaptogenesis and neuronal repair. (Alkon, D. L., et al., Protein synthesis required for long-term memory is induced by PKC activation on days before associative learning. Proc Natl Acad Sci USA, 2005. 102(45): p. 16432-7; Bonini, J. S., et al., Inhibition of PKC in basolateral amygdala and posterior parietal cortex impairs consolidation of inhibitory avoidance memory. Pharmacol Biochem Behav, 2005. 80(1): p. 63-7; Etcheberrigaray, R., et al., Therapeutic effects of PKC activators in Alzheimer's disease transgenic mice. Proc Natl Acad Sci USA, 2004. 101(30): p. 11141-6) In particular, PKC delta (PKCδ) has been implicated in memory, neuronal survival and proliferation. (Conboy, L., et al., Curcumin-induced degradation of PKCdelta is associated with enhanced dentate NCAM PSA expression and spatial learning in adult and aged Wistar rats. Biochem Pharmacol, 2009. 77(7): p. 1254-65; Ferri, P., et al., alpha-Tocopherol affects neuronal plasticity in adult rat dentate gyms: the possible role of PKCdelta. J Neurobiol, 2006. 66(8): p. 793-810; Fujiki, M., et al., Role of protein kinase C in neuroprotective effect of geranylgeranylacetone, a noninvasive inducing agent of heat shock protein, on delayed neuronal death caused by transient ischemia in rats. J Neurotrauma, 2006. 23(7): p. 1164-78)

PKCδ plays a central role in apoptosis. Various lines of evidence point to the role of protein kinase C delta (PKCδ) isoforms in regulating apoptosis in the brain. (Blass, M., et al., Tyrosine phosphorylation of protein kinase C delta is essential for its apoptotic effect in response to etoposide. Mol Cell Biol, 2002. 22(1): p. 182-95; Brodie, C. and P. M. Blumberg, Regulation of cell apoptosis by protein kinase c delta. Apoptosis, 2003. 8(1): p. 19-27) PKCδ is a substrate for and activator of caspase-3, indicating a positive feedback loop between the two enzymes. In response to apoptotic stimuli, PKCδI is proteolytically cleaved at the V3 hinge domain by caspase 3. (Emoto, Y., et al., Proteolytic activation of protein kinase C delta by an ICE-like protease in apoptotic cells. Embo J, 1995. 14(24): p. 6148-56; Ghayur, T., et al., Proteolytic activation of protein kinase C delta by an ICE/CED 3-like protease induces characteristics of apoptosis. J Exp Med, 1996. 184(6): p. 2399-404; Kohtz, J. D., et al., Protein-protein interactions and 5′-splice-site recognition in mammalian mRNA precursors. Nature, 1994. 368: p. 119-124) The release of the catalytically active fragment induces nuclear fragmentation and apoptosis in various cell types, including dopaminergic neuronal cell lines. (Anantharam, V., et al., Caspase-3-dependent proteolytic cleavage of protein kinase Cdelta is essential for oxidative stress-mediated dopaminergic cell death after exposure to methylcyclopentadienyl manganese tricarbonyl. J Neurosci, 2002. 22(5): p. 1738-51) Furthermore, caspase-induced apoptosis is blocked by inhibiting the catalytic fragment of PKCδI. (Reyland, M. E., et al., Protein kinase C delta is essential for etoposide-induced apoptosis in salivary gland acinar cells. J Biol Chem, 1999. 274(27): p. 19115-23) The V3 region of PKCδ contains the caspase-3 recognition sequence, DXXD (P4-P1)/X. The cleavage and activation of PKCδ sets up a positive feedback loop that impinges upon upstream components of the death effector pathway, thereby amplifying the caspase cascade and helping cells commit to apoptosis. (Denning, M. F., et al., Caspase activation and disruption of mitochondrial membrane potential during UV radiation-induced apoptosis of human keratinocytes requires activation of protein kinase C. Cell Death Differ, 2002. 9(1): p. 40-52; Sitailo, L., S. Tibudan, and M. F. Denning, Bax activation and induction of apoptosis in human keratinocytes by protein kinase C delta catalytic domain. Jour of Investigative Dermatology, 2004: p. 1-10; Sitailo, L. A., S. S. Tibudan, and M. F. Denning, The protein kinase C delta catalytic fragment targets Mcl-1 for degradation to trigger apoptosis. J Biol Chem, 2006. 281(40): p. 29703-10)

Other studies, however, implicated PKCδ in cell-survival and anti-apoptotic effects. In granulosa and PC12 cells, apoptosis is prevented by basic fibroblast growth factor acting through a PKCδ pathway. (Peluso, J. J., A. Pappalardo, and G. Fernandez, Basic fibroblast growth factor maintains calcium homeostasis and granulosa cell viability by stimulating calcium efflux via a PKC delta-dependent pathway. Endocrinology, 2001. 142(10): p. 4203-11) In human neutrophils, PKCδ participates in the anti-apoptotic effects of TNFα. (Kilpatrick, L. E., et al., A role for PKC-delta and PI 3-kinase in TNF-alpha-mediated antiapoptotic signaling in the human neutrophil. Am J Physiol Cell Physiol, 2002. 283(1): p. C48-57) PKCδ also has anti-apoptotic effects in glioma cells infected with a virulent strain of Sindbis virus. (Zrachia, A., et al., Infection of glioma cells with Sindbis virus induces selective activation and tyrosine phosphorylation of protein kinase C delta. Implications for Sindbis virus-induced apoptosis. J Biol Chem, 2002. 277(26): p. 23693-701) In human breast tumor cell lines, PKCδ acts as a pro-survival factor. McCracken, M. A., et al., Protein kinase C delta is a prosurvival factor in human breast tumor cell lines. Mol Cancer Ther, 2003. 2(3): p. 273-81) Thus, PKCδ has dual effects as a mediator of apoptosis and as an anti-apoptosis effector. Therefore, its splice variants may be a switch that determines cell survival and fate.

The expression of PKCδ splice variants is species-specific. PKCδI is ubiquitous in all species. PKCδII, -δIV, -δV, -δVI, and -δVII are present in mouse tissues, PKCδIII is present in rats, and PKCδVIII is present in humans. (Sakurai, Y., et al., Novel protein kinase C delta isoform insensitive to caspase-3. Biol Pharm Bull, 2001. 24(9): p. 973-7; Kawaguchi, T., et al., New PKCdelta family members, PKCdeltaIV, deltaV, deltaVI, and deltaVII are specifically expressed in mouse testis. FEBS Lett, 2006. 580(10): p. 2458-64; Ueyama, T., et al., cDNA cloning of an alternative splicing variant of protein kinase C delta (PKC deltaIII), a new truncated form of PKCdelta, in rats. Biochem Biophys Res Commun, 2000. 269(2): p. 557-63) The inventors have shown that PKCδII and PKCδVIII function as pro-survival proteins; the functions of the other isoforms are not yet established. PKCδII is the mouse homolog of human PKCδVIII; both are generated by alternative 5′ splice site usage, and their transcripts share >94% sequence homology.

Alternative Splicing:

An important mechanism of regulating gene expression is alternative splicing which dramatically expands the coding capacity of a single gene to produce different proteins with distinct functions. (Hastings, M. L. and A. R. Krainer, Pre-mRNA splicing in the new millennium. Curr Opin Cell Biol, 2001. 13(3): p. 302-9) Alternative splicing occurs in more than 85% of genes and is the single most powerful step in gene expression to diversify the genomic repertoire. (Modrek, B. and C. Lee, A genomic view of alternative splicing. Nat Genet, 2002. 30(1): p. 13-9)

Divergence observed in gene expression due to alternative splicing may be tissue-specific, developmentally regulated or hormonally regulated. (Hiroyuki Kawahigashi, Y. H., Akira Asano, Masahiko Nakamura, A cis acting regulatory element that affects the alternative splicing of a muscle-specific exon in the mouse NCAM gene. BBA, 1998. 1397: p. 305-315; Libri, D., A. Piseri, and M. Y. Fiszman, Tissue specific splicing in vivo of the beta tropomyosin gene: dependence on an RNA secondary structure. Science, 1991. 252: p. 1842-1845; A. F. Muro, A. I., F. E. Baralle, Regulation of the fibronectin EDA exon alternative splicing. Cooperative role of exonic enhancer element and the 5′ splicing site. FEBS Letters, 1998. 437: p. 137-141; Du, K., et al., HRS/SRp40-mediated inclusion of the fibronectin E111B exon, a Possible cause of increased EIIIB expression in proliferating liver. MCB, 1997. 17: p. 4096-4104; Chalfant, C. E., et al., Regulation of alternative splicing of protein kinase Cbeta by insulin. Journal of Biological Chemistry, 1995. 270: p. 13326-13332; Patel, N. A., et al., Insulin regulates protein kinase CbetaII alternative splicing in multiple target tissues: development of a hormonally responsive heterologous minigene. Mol Endocrinol, 2004. 18(4): p. 899-911)

Alternative splicing can occur through various mechanisms such as exon skipping, exon inclusion, alternative 3′ splice site usage, alternative 5′ splice site usage, or alternative polyadenylation site usage. For efficient splicing, most introns require cis elements comprising of a conserved 5′ splice site (AG↓GUpu), a branch point (BP) sequence (CupuApy) followed by a polypyrimidine tract and a 3′ splice site (pyAG↓puN). The spliceosome catalyzes the pre-mRNA splicing reaction within a large multicomponent ribonucleoprotein complex. Signals exist in the pre-mRNA as auxiliary cis-elements that recruit trans-acting factors to promote alternative splicing. Exonic or intronic splicing enhancers (ESE, ISE) often bind the serine-arginine rich nuclear factors—SR proteins—to promote the choice of splice sites in the pre-mRNA. The binding of SR proteins to exonic or intronic sites defines splice site choice. (Patel, N. A., S. S. Song, and D. R. Cooper, PKCdelta alternatively spliced isoforms modulate cellular apoptosis in retinoic acid-induced differentiation of human NT2 cells and mouse embryonic stem cells. Gene Expr, 2006. 13(2): p. 73-84)

SC35, also known as SFRS2 or SRp30b, is a member of the nuclear serine-arginine rich (SR) splicing proteins family and functions as a splicing enhancer. (Liu, H. X., et al., Exonic splicing enhancer motif recognized by human SC35 under splicing conditions. Mol Cell Biol, 2000. 20(3): p. 1063-71) SC35 has an N-terminal RNA recognition motif (RRM) domain and a C-terminal arginine/serine rich (RS) domain. The RRM domain is the region where it interacts and binds to the target pre-mRNA while the RS domain is highly phosphorylated. SC35 has been shown to be involved in pathways that regulate genomic stability and cell proliferation during mammalian organogenesis. (Xiao, R., et al., Splicing Regulator SC35 Is Essential for Genomic Stability and Cell Proliferation during Mammalian Organogenesis. Mol Cell Biol, 2007) SC35 also plays a role in aberrant splicing of tau exon 10 in Alzheimer's disease as well as in splicing of neuronal acetylcholinesterase mRNA. (Hernandez, F., et al., Glycogen synthase kinase-3 plays a crucial role in tau exon 10 splicing and intranuclear distribution of SC35. Implications for Alzheimer's disease. J Biol Chem, 2004. 279(5): p. 3801-6; Meshorer, E., et al., SC35 promotes sustainable stress-induced alternative splicing of neuronal acetylcholinesterase mRNA. Mol Psychiatry, 2005. 10: p. 985-997)

RA and Alternative Splicing:

Alternative splicing in neurons is now considered to be a central phenomenon in development, evolution and survival of neurons. (Lee, C. J. and K. Irizarry, Alternative splicing in the nervous system: an emerging source of diversity and regulation. Biol Psychiatry, 2003. 54(8): p. 771-6) Interestingly, current literature suggests an emerging role of retinoic acid in alternative splicing events. In P19 embryonal carcinoma stem cells, during RA-induced differentiation the co-activator CoAA rapidly switches to its dominant negative splice variant CoAM. (Yang, Z. Z., et al., Switched alternative splicing of oncogene CoAA during embryonal carcinoma stem cell differentiation. Nuc Acids Res, 2007. 35(6): p. 1919-1932) In the same cells, the splicing pattern of the delta isoform of CaM kinase is also changed with RA-induced differentiation. (Donai, H., et al., Induction and alternative splicing of delta isoform of Ca(+2)/calmodulin-dependent protein kinase II during neural differentiation of P19 embryonal carcinoma cells and brain development. Brain Res Mol Brain Res, 2000. 85(1-2): p. 189-199) RA alters the expression of a dynamic set of regulatory genes at the early stages of differentiation. (Spinella, M. J., et al., Retinoid Target Gene Activation during Induced Tumor Cell Differentiation: Human Embryonal Carcinoma as a Model. J. Nutr., 2003. 133(1): p. 273S-276) The inventors have shown that RA regulates alternative splicing of PKCδ isoforms in NT2 cells.

Links Between Coupling of Transcription and Splicing:

Recent evidence indicates a high degree of co-ordination in time and space between transcription machinery and assembly of the spliceosome. This assembly of the spliceosome influences pre-mRNA alternative splicing and splice site selection. Pre-mRNA splicing begins co-transcriptionally when the nascent RNA is still attached to DNA by RNA polymerase II. (Neugebauer, K. M., On the importance of being co-transcriptional. J Cell Sci, 2002. 115(Pt 20): p. 3865-71; Neugebauer, K. M., Please hold—the next available exon will be right with you. Nat Struct Mol Biol, 2006. 13(5): p. 385-6) Functional links exist between transcription and splicing as reviewed extensively by Kornblihtt et al. (Kornblihtt, A. R., et al., Multiple links between transcription and splicing. Rna, 2004. 10(10): p. 1489-98) The C-terminal domain (CTD) of RNA polymerase II plays a central role in linking transcription with the splicing machinery. (Nogues, G., et al., Control of alternative pre-mRNA splicing by RNA Pol II elongation: faster is not always better. IUBMB Life, 2003. 55(4-5): p. 235-41) It has been proposed that the CTD of RNA polymerase II facilitates recruitment of co-activators and splicing factors. Phosphorylated CTD can recruit splicing factors and affect splicing decisions. (Zeng, C., et al., Dynamic relocation of transcription and splicing factors dependent upon transcriptional activity. Embo J, 1997. 16(6): p. 1401-12) Further, splicing factors have been shown to have a stimulatory effect on transcription elongation. (Fong, Y. W. and Q. Zhou, Stimulatory effect of splicing factors on transcriptional elongation. Nature, 2001. 414(6866): p. 929-33)

Transcription by RNA polymerase II involves recruiting splicing enhancers (such as SR proteins) to the transcription site. It has been demonstrated that RNA polymerase II forms a large complex with factors associated with splicing. (Millhouse, S, and J. L. Manley, The C-terminal domain of RNA polymerase II functions as a phosphorylation-dependent splicing activator in a heterologous protein. Mol Cell Biol, 2005. 25(2): p. 533-44; Robert, F., et al., A human RNA polymerase II-containing complex associated with factors necessary for spliceosome assembly. J Biol Chem, 2002. 277(11): p. 9302-6; Du, L. and S. L. Warren, A functional interaction between the carboxy-terminal domain of RNA polymerase II and pre-mRNA splicing. J Cell Biol, 1997. 136(1): p. 5-18; Kim, E., et al., Splicing factors associate with hyperphosphorylated RNA polymerase II in the absence of pre-mRNA. J Cell Biol, 1997. 136(1): p. 19-28; Mortillaro, M. J., et al., A hyperphosphorylated form of the large subunit of RNA polymerase II is associated with splicing complexes and the nuclear matrix. Proc Natl Acad Sci USA, 1996. 93(16): p. 8253-7) It is not obligatory for all alternatively spliced genes to be regulated co-transcriptionally but the physical association or complex formation by RNA polymerase II and trans-factors (both involved in transcription and post-transcriptional processes) facilitates efficient transcription and splicing. The complex readily provides the factors required for post-transcriptional alternative splicing thereby increasing the efficiency.

Steroid hormone receptors which belong to the nuclear receptors superfamily have been shown to control alternative splicing of the transcripts of their transcriptional target genes. Further, it has been demonstrated that nuclear receptors induce formation of transcriptional complexes that stimulate transcript production and control the nature of the spliced variants produced from these genes. (Auboeuf, D., et al., Differential recruitment of nuclear receptor coactivators may determine alternative RNA splice site choice in target genes. Proc Natl Acad Sci USA, 2004. 101(8): p. 2270-4; Auboeuf, D., et al., Coordinate regulation of transcription and splicing by steroid receptor coregulators. Science, 2002. 298(5592): p. 416-9)

Preliminary computer analyses of the PKC promoter in the laboratory have shown the presence of RAREs. The cooperative role of RARE in promoter region and post-transcriptional alternative splicing of PKC has not yet been elucidated. Prior studies have shown that RA induces the expression of PKCα gene through transcriptional stimulation of its promoter. (Niles, R. M., Vitamin A (retinoids) regulation of mouse melanoma growth and differentiation. J Nutr, 2003. 133(1): p. 282S-286S) McGrane et al have demonstrated that RNA polymerase II associates with the retinoic-acid response element (RARE) on the promoter of phosphoenolpyruvate carboxykinase (PEPCK), a RA-responsive gene. (McGrane, M. M., Vitamin A regulation of gene expression: molecular mechanism of a prototype gene. J Nutr Biochem, 2007; Scribner, K. B. and M. M. McGrane, RNA polymerase II association with the phosphoenolpyruvate carboxykinase (PEPCK) promoter is reduced in vitamin A-deficient mice. J Nutr, 2003. 133(12): p. 4112-7) It has been demonstrated that RNA pol II associates tightly with SC35 in MDCK cells. (Bregman, D. B., et al., Transcription-dependent redistribution of the large subunit of RNA polymerase II to discrete nuclear domains. J Cell Biol, 1995. 129(2): p. 287-98)

The inventors have discovered a splice variant of human PKCδ, PKCδVIII which is highly expressed in the brain. (Jiang, K., et al., Identification of a Novel Antiapoptotic Human Protein Kinase C delta Isoform, PKCdeltaVIII in NT2 Cells. Biochemistry, 2008. 47(2): p. 787-797) PKCδ is alternatively spliced into PKCδI, which is apoptotic, and PKCδVIII, which promotes survival (Patel, N. A., S. Song, and D. R. Cooper, PKCdelta alternatively spliced isoforms modulate cellular apoptosis in retinoic-induced differentiation of human NT2 cells and mouse embryonic stem cells. Gene Expression, 2006. 13(2): p. 73-84). Human PKCδI mRNA sequence coding for 674 amino acids has a molecular mass of 78 kDa while PKCδVIII mRNA sequence codes for 705 amino acids and has a molecular mass of ˜81 kDa. PKCδVIII has an insertion of 93 bp (i.e. 31 amino acids) in its caspase 3-recognition sequence −DMQD. PKCδVIII is resistant to cleavage by caspase-3. The inventors demonstrate that RA increases the expression of PKCδVIII by regulating alternative splicing. Splicing factors are key determinants of alternative splicing. RA activated the splicing factor SC35, which in concert with cis-elements up-regulated PKCδVIII expression. In vitro splicing assays were performed to measure the influences of SC35 on the efficiency of PKCδ pre-mRNA splice site selection. These assays allow for manipulation of splicing reactions to study its mechanism and regulation by retinoic acid. It was found that over-expression of PKCδVIII decreases cellular apoptosis. siRNA mediated knockdown of PKCδVIII further demonstrated that PKCδVIII functions as an anti-apoptotic protein. Increased expression of PKCδVIII shields cells from etoposide-mediated apoptosis.

### SUMMARY OF INVENTION

Vitamin A metabolite, all-trans-retinoic acid (RA), induces cell growth, differentiation, and apoptosis where it is involved in the caspase-3 mediated apoptotic pathway. Cleavage of PKCδI by caspase-3 releases a catalytically-active C-terminal fragment which is sufficient to induce apoptosis. RA has an emerging role in gene regulation and alternative splicing events. Protein kinase Cδ (PKCδ), a serine/threonine kinase, has a role in cell proliferation, differentiation, and apoptosis. The inventors previously discovered an alternatively spliced variant of human PKCδ, PKCδVIII (Genbank accession number DQ516383) that functions as a pro-survival protein and whose expression levels are highest in the brain. Expression of PKCδVIII was confirmed by real time RT-PCR analysis. Using in vivo and in vitro assays the inventors have demonstrated that PKCδVIII is resistant to caspase-3 cleavage.

RA regulates the splicing and expression of PKCδVIII via utilization of a downstream 5′ splice site of exon 10 on PKCδ pre-mRNA. Overexpression and knockdown of the splicing factor SC35 (i.e. SRp30b) indicated that it is involved in PKCδVIII alternative splicing. To identify the cis-elements involved in 5′ splice site selection we cloned a minigene, which included PKCδ exon 10 and its flanking introns in the pSPL3 splicing vector. Alternative 5′ splice site utilization in the minigene was promoted by RA. Further, co-transfection of SC35 with PKCδ minigene promoted selection of 5′ splice site II. Mutation of the SC35 binding site in the PKCδ minigene abolished RA-mediated utilization of 5′ splice II. RNA binding assays demonstrated that the enhancer element downstream of PKCδ exon 10 is a SC35 cis-element. The inventors found that SC35 is pivotal in RA-mediated PKCδ pre-mRNA alternative splicing.

It was also found that over-expression of PKCδVIII increased the expression of pro-survival proteins Bcl2 and Bcl-xL. This indicates that PKCδVIII mediates its effects via Bcl2 and Bcl-xL. PKCδVIII holds the switch for the cell to undergo cell death or shield the cell from apoptosis (programmed cell death). Increased expression of PKCδVIII in neurons is indicative of cancer while greatly decreased expression of PKCδVIII in hypothalamus or temporal lobe of brain is indicative of early stages of AD. Using these data, PKCδVIII can serve as a biomarker for neurodegenerative diseases such as Alzheimer's disease as well as neuronal cancers.

In one embodiment of the invention, a method of predicting neurodegenerative disease is presented. The method comprises: obtaining the expression levels of PKCδVIII in a test tissue and comparing the expression levels of PKCδVIII to a predetermined control expression level, wherein a decrease in expression levels indicates neurodegenerative disease. The neurodegenerative disease can be selected from the group consisting of Alzheimer's disease, Parkinson's disease, Huntington's disease, dementia, amyotrophic lateral sclerosis, and multiple sclerosis.

In another embodiment, a method of predicting neuronal metastases is presented. The method is comprised of: obtaining the expression levels of PKCδVIII in a test tissue and comparing the expression levels of PKCδVIII to a predetermined control expression level, wherein an increase in expression levels indicates neuronal metastases. The neuronal metastases can be selected from the group consisting of gliomas and neuroblastomas.

In a further embodiment, a method of modulating expression of PKCδ isozymes in cells is presented comprising administering an effective amount of a compound that affects the splicing enhancer SC35. The compound can increase levels of splicing enhancer SC35. The compound can increase expression of PKCδVIII. The compound can be all-trans retinoic acid and can be administered at about 10 μM for about 24 hours.

A further embodiment includes a method of modulating neuronal cell survival in a subject comprising modulating levels of PKCδ isozymes. The neuronal cell survival can be increased by increasing levels of PKCδVIII. The level of PKCδVIII can be increased by administering an effective amount of retinoic acid to the cells. The level of PKCδVIII can be increased by increasing amounts of splicing enhancer SC35 in the cell.

A further embodiment encompasses a method of modulating apoptosis in cells comprising modulating levels of PKCδ isozymes. Apoptosis may be decreased by increasing levels of PKCδVIII. The level of PKCδVIII can be increased by administering an effective amount of retinoic acid to the cells. The level of PKCδVIII can be increased by increasing amounts of splicing enhancer SC35 in the cell. The apoptosis that is modulated can be etoposide-mediated apoptosis.

### BRIEF DESCRIPTION OF THE DRAWINGS

For a fuller understanding of the invention, reference should be made to the following detailed description, taken in connection with the accompanying drawings, in which:

FIG. 1 is a series of images illustrating the alternative splice site in human PKC δ. (a) schematic of alternative 5′ splice site selection in human PKCδ pre-mRNA exon 10 that results in the generation of PKCδI mRNA and PKCδVIII mRNA, which differ by about 93 bp in the V3 hinge region. RA promotes expression of PKCδVIII mRNA. SSI: 5′ splice site I; SSII: 5′ splice site II. (b) schematic of the primers specific for PKC δI and PKC δVIII used in real time RT-PCR such that they span the exon-exon boundaries. (c) primary human neuronal cells from hippocampus were treated with or without RA (about 10 μM) for about 24 h. Total RNA was extracted, and real time RT-PCR analysis using SYBR green was performed in triplicate and repeated three times in separate experiments. The absolute mRNA expression of PKCδI and PKCδVIII transcripts normalized to GAPDH are shown. PKCδVIII expression increases significantly following about 24 h of RA treatment; ***, p<0.0001 (by two-tailed Student's t test).

FIG. 2 is a series of images indicating the expression of PKCδVIII. (a) an image showing that both PKCδI and PKCδVIII were detected and the levels of PKCδVIII increased with retinoic acid treatment. Primary neuronal cells from hippocampus were treated with RA for about 24 h. Total RNA was extracted and RT-PCR performed with primers described in FIG. 1 which detect both PKCδI and PKCδVIII simultaneously. (b) an image showing that the expression of PKCδVIII is tissue-specific with the highest levels seen in the fetal brain. Human fetal tissue specific cdNAs were used in PCR analysis using PKCδVIII-specific primers. (i) Liver (ii) kidney (iii) heart (iv) spleen (v) brain. About five percent products were separated on PAGE and detected by silver nitrate staining

FIG. 3 is a series of images illustrating PKCδVIII levels in Alzheimer's disease patients as well as in glioma and neuroblastoma cell lines. (a) an image showing PKCδVIII expression is dramatically decreased in Alzheimer's disease patients compared to their matched controls while increased PKCδVIII levels are observed in glioma and neuroblastoma cell lines. Total RNA was isolated from brain sections from Alzheimer's disease (AD) patients (#3-6) and matched control patients (#1-2). TL: temporal lobe; HP: hippocampus; RT-PCR was performed using human PKCδ primers. (b) an image showing PKCδVIII expression is dramatically decreased in Alzheimer's disease patients compared to their matched controls while increased PKCδVIII levels are observed in glioma and neuroblastoma cell lines. Total RNA was extracted RT-PCR performed with primers specific for PKCδVIII. Lanes: M: Marker; 1: NT2+RA; 2: breast cancer cell line MDA-468-MB; 3: LnCapandrogen dependent prostrate cancer; 4: glioma cell lines U-138MG; 5: glioma cell lines T98G; 6: Human neuroblastoma cells BE(2)-C. (c) an image showing PKCδVIII expression is dramatically decreased in Alzheimer's disease patients compared to their matched controls while increased PKCδVIII levels are observed in glioma and neuroblastoma cell lines. Total RNA was isolated from brain sections from Alzheimer's disease (AD) patients and matched control patients. TL: temporal lobe; HP: hippocampus; RT-PCR was performed using human PKCδ primers. Graph represents percent exon inclusion calculated as PKXδVIII/(δVIII+δI)×100 in control and AD samples and is representative of about 30 samples analyzed.

FIG. 4 is a 3D profile of the results from the apoptosis micro-array. The graph represents an average of control and RA (1 day) samples carried out in triplicate. The average ΔCt=Ct(gene of interest)−Ct(housekeeping gene). The expression level ((2̂(−ΔCt)) of each gene in the control sample versus the test (RA) sample is calculated followed by the student's t-test and is represented as the fold regulation. Inset shows PCR using Bcl-2 primers performed on same sample.

FIG. 5 is a series of images depicting that PKCδVIII promotes the expression of Bcl-2. (a) an image illustrating that PKCδVIII promotes the expression of Bcl-2. Bcl-2 expression is increased concomitantly with an increase in PKCδVIII expression. Two μg of PKCδVIII_GW was transiently transfected in NT2 cells for about 48 h. Total RNA was extracted and RT-PCR was performed using human PKCδ, Bcl-2, Bcl-x or GAPDH primers as indicated. About five percent of the products were separated by PAGE and silver stained for visualization. (b) an image illustrating that PKCδVIII promotes the expression of Bcl-2. Western blot analysis was performed with antibodies as indicated.

FIG. 6 is a series of images illustrating the detection of SR proteins involved in RA-mediated PKXδVIII expression. NT2 cells were treated with RA (about 10 μM) for about 24 h or without RA (control), and Western blot analysis was performed on whole cell lysates using (a) mAb104 antibody that detects all SR proteins and (b) specific antibodies as indicated in the figure. Molecular masses are indicated (kDa). Gels are representative of three separate experiments, and results indicate that SC35 may be involved in increased expression of PKXδVIII by RA. Results demonstrate an increase in SC35 levels concurrent with an increase in PKXδVIII expression upon RA treatment.

FIG. 7 is a series of images illustrating that SC35 but not SF2/ASF promotes PKCδVIII expression. (a) schematic of primer positions used in PCR amplification. These primers detect PKCδI and PKCδVIII simultaneously. (b) NT2 cells were transfected with about 2 μg of SC35 or SF2/ASF or treated with RA (about 10 μM) for about 24 h. Total RNA was extracted, and RT-PCR was performed using human PKCδ primers as shown above. About 5% of the products were separated by PAGE and silver stained for visualization. The graph represents percent exon inclusion calculated as PKCδVIII/(δVIII/δI)×100 in these samples and is representative of mean±S.E. in three experiments. (c) whole cell lysates were extracted from NT2 cells transfected with about 2 μg of SC35 or SF2/ASF. Western blot analysis was performed using specific antibodies as indicated in the figure. The experiments were repeated three times with similar results. (d) increasing amounts of SC35 (about 0 to about 2 μg) were transfected into NT2 cells and treated with or without RA (about 10 μM, about 24 h). Total RNA was extracted and RT-PCR was performed using human PKCδ primers as shown above. About 5% of the products were separated by PAGE and silver stained for visualization. Graph represents percent exon inclusion calculated as PKCδVIII/(δVIII/δI)×100 in these samples and is representative of mean±S.E. in three experiments. (e) simultaneously, Western blot analysis was performed on whole cell lysates extracted from NT2 cells transfected with about 0-2 μg of SC35, using antibodies as indicated within the figure. The graph represents four experiments performed separately and represents PKCδVIII densitometric units normalized to GAPDH as mean±S.E. The triangle in the graphs indicates increasing amounts of SC35. Results indicate that SC35 promotes PKCδVIII expression in a dose-dependent manner thereby mimicking the RA response.

FIG. 8 is a series of images depicting knockdown of SC35 inhibits RA-mediated increased expression of PKCδVIII. Increasing amounts of SC35 siRNA (about 0-about 150 nM) were transfected into NT2 cells. Scrambled siRNA was used as a control (con siRNA). Post-transfection, cells were treated with or without RA (about 10 μM, about 24 h). (a) total RNA was extracted, and RT-PCR was performed using human PKCδ primers as shown above. About 5% of the products were separated by PAGE and silver stained for visualization. Graph represents percent exon inclusion calculated as PKCδVIII/(δVIII/δI)×100 in these samples and is representative of mean±S.E. in three experiments. (b) simultaneously, whole cell lysates were collected, and Western blot analysis was performed using antibodies as indicated. Graph represents four experiments performed separately and expressed as mean±S.E. of densitometric units. The triangle in the graphs indicates increasing amounts of SC35 siRNA. Results indicate that knockdown of SC35 inhibits RA-mediated increased expression of PKCδVIII.

FIG. 9 is a series of images depicting analysis of putative cis-elements and ASO. (a) schematic of position of ASOs on PKCδ pre-mRNA. The putative SC35 cis-element lies between 5′ splice site I and II of PKCδ exon 10. SSI: 5′ splice site I; SSII: 5′ splice site II. (b) ASOs were transfected into NT2 cells and after overnight incubation cells were treated with or without RA (about 10 μM, about 24 h). The gel represents experiments conducted with scrambled ASO (control), ASO 81 (corresponding to putative SC35 binding site) and ASO 80, which is in close proximity to ASO81. Total RNA was extracted and RT-PCR performed using PKCδVIII-specific primers. About 5% products were separated on PAGE and detected by silver nitrate staining. The graph indicates PKCδVIII densitometric units normalized to GAPDH and is representative of mean±S.E. in three separate experiments. Results indicate that ASO81, which corresponds to the putative SC35 cis-element, inhibits RA-mediated increased expression of PKCδVIII.

FIG. 10 is a series of images depicting minigene analysis demonstrates that RA promotes utilization of 5′ splice site II on PKCδ exon 10 pre-mRNA. (a) schematic represents PKCδ pre-mRNA exon 10 and flanking introns cloned into pSPL3 splicing vector between the SD and SA exons. The resulting minigene is referred to as pSPL3_PKCδ minigene. Arrows indicate position of primers used in RT-PCR analysis. (b) pSPL3_PKCδ minigene and pSPL3 empty vector were transfected overnight, and then the cells were treated with or without about 10 μM RA for about 24 h. Total RNA was extracted and RT-PCR performed using primers as described above. Expected products are SD-SA: constitutive splicing; SSI: usage of 5′ splice site I; SSII: usage of 5′ splice site II. (c) About 2 μg of SC35 or SF2/ASF was co-transfected along with the pSPL3_PKCδ splicing minigene. In separate wells, 10 μM RA was added for 24 h. Total RNA was extracted and RT-PCR performed using PKCδ exon 10 and SA primers as shown in the schematic. SSI: usage of 5′ splice site I; SSII: usage of 5′ splice site II. (d) SC35 siRNA (about 100 nM) or scrambled siRNA was co-transfected with pSPL3_PKCδ minigene. 10 μM RA was added to wells as indicated. Total RNA was extracted and RT-PCR performed using PKCδ exon 10 and SA primers as shown above in c. About 5% of the products were separated by PAGE and silver stained for visualization. Graphs represent percent exon inclusion calculated as SS II/(SS II+SSI)×100 in the samples and are representative of four experiments performed separately. These results demonstrate that co-transfection of SC35 with the pSPL3_PKCδ minigene promotes utilization of 5′ splice site II. Further, RA is unable to promote utilization of 5′ splice site II on PKCδVIII pre-mRNA in the absence of SC35.

FIG. 11 is a series of images depicting mutation of putative SC35 binding site inhibits RA-mediated utilization of 5′ splice site II utilization on the minigene. (a) schematic of the position and sequence of the putative SC35 cis-element on the pSPL3_PKCδ splicing minigene. Arrows indicate the position of primers used in PCR analysis. Putative SC35 binding site ggccaaag (SEQ ID No: 17) was mutated to tagcccaga (SEQ ID No: 18) on the minigene. (b) resulting mutated minigene pSPL3_PKCδ** was transfected into NT2 cells. In separate wells, the mutated minigene pSPL3_PKCδ** was co-transfected with either about 2 μg of SC35 or SF2/ASF. The original pSPL3_PKCδ splicing minigene was also transfected in a separate well. After overnight transfection, NT2 cells were treated with or without about 10 μM RA for about 24 h. Total RNA was extracted and RT-PCR performed using primers for PKCδ exon 10 sense and SA antisense as shown. About 5% of the products were separated by PAGE and silver stained for visualization. SSI: usage of 5′ splice site I; SSII: usage of 5′ splice site II. Graph represents percent exon inclusion calculated as SS II/(SS II+SSI)×100 and is representative of three experiments performed separately. Results indicate that mutation of the enhancer element ggccaaag abolishes the ability of RA or SC35 to promote utilization of 5′ splice site II on PKCδ splicing minigene.

FIG. 12 is a series of images depicting gel mobility assays of F1 and mutated F1 with purified recombinant SC35. (a) schematic representation of the position of PKCδtranscripts F1, F1m and F2 used in the gel binding assays. F1 contains exon 10 and 120 bp of flanking 5′ sequence, which includes the enhancer sequence ggccaaag; schematic also indicates its position on the PKCδ pre-mRNA. F1m is the same as F1 with the enhancer sequence mutated to tagcccata. F2 transcript contains PKCδ10 exon only. (b) the biotin-labeled in vitro transcribed RNA sequences were incubated with recombinant SC35 at about 30° C. for about 20 min. The complex was run on an 8% polyacrylamide gel and transferred to a nylon membrane. Western blot analysis was performed using an avidin-HRP conjugate. Lanes are 1: F1; 2: F1+SC35; 3: F2+SC35; 4: F1m; 5: F1m+SC35. The bracket indicates RNA-protein complex. The gel represents four experiments performed separately. Results indicate that ggccaaag is an SC35 cis-element on PKCδ pre-mRNA.

FIG. 13 is a series of images demonstrating a schematic for generating templates for in vitro transcription. (a) The first splicing template was used to generate preliminary data. The forward primer is on the 3′ intron such that the branch point and 3′ splice site of exon 10 is included in the product. The reverse primer is on the intron such that the 5′ splice site of exon 11 is included. The product length is about 562 bp. The forward primer has Xho I site and the reverse primer has Not 1 site (bold text on primer sequence) to enable cloning in the correct orientation into the MCS of the vector.

(SEQ ID No: 19)
Forward primer: 5′ CCTTCTCGAGCTGGGCTGGGAGTTCTG 3′
(SEQ ID No: 20)
Reverse primer: 5′ CCCACCTCAGCCACGCGGCCGCCTAA 3′

(b) The second splicing template is shown in 2 steps to eliminate the extra intronic sequences between the 5′ splice II of exon 10 and exon 11. The steps are as follows: (i) Two PCR products will be generated. The sequence in bold on the primers below is the KpnI site which is not present on the PKCδ sequence and will aid to orient the products correctly for ligation. First product will be amplified using the same forward primer as described above for template 1. The reverse primer will be 5′ CGGTGGTTCCTTCCCCGGTACCTG 3′. (SEQ ID No: 21) The product length is about 269 bp. The next PCR product will be amplified using the forward primer 5′ TCGGTACCGGGCAGACAACAGTGG 3′. (SEQ ID No: 22) The product length is about 181 bp. The reverse primer will be the same as described above for template 1. (ii) Ligation of the products: The two PCR products will be then digested with KpnI to produce compatible ends for ligation using DNA ligase (Stratagene).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed in the invention. The upper and lower limits of these smaller ranges may independently be excluded or included within the range. Each range where either, neither, or both limits are included in the smaller ranges are also encompassed by the invention, subject to any specifically excluded limit in the stated range. Where the stated range includes one or both of the limits, ranges excluding either or both of those excluded limits are also included in the invention.

The kinases of the present invention may serve as biomarkers for: (1) the diagnosis of disease; (2) the prognosis of diseases (e.g. monitoring disease progression or regression from one biological state to another; (3) the determination of susceptibility or risk of a subject to disease; or (4) the evaluation of the efficacy to a treatment for disease. For the diagnosis of disease, the level of the specific kinase isozyme in the subject can be compared to a baseline or control level in which if the level is above the control level, a certain disease is implicated whereas if the level is below the control level, a different disease is implicated. The prognosis of disease can be assessed by comparing the level of the specific kinase biomarker at a first timepoint to the level of the biomarker at a second timepoint which occurs at a given interval after the first timepoint. The evaluation of the efficacy of the treatment for a disease can be assessed by comparing the level of the specific kinase biomarker at a first timepoint before administration of the treatment to the level of the biomarker at a second timepoint which occurs at a specified interval after the administration of the treatment.

The term “subject” as used herein describes an animal, preferably a human, to whom treatment is administered.

The term “biomarker” is used herein to refer to a molecule whose level of nucleic acid or protein product has a quantitatively differential concentration or level with respect to an aspect of a biological state of a subject. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a prepropeptide, a propeptide, a mature peptide or a secreted peptide of the biomarker may be measured. A biomarker can be used either solely or in conjunction with one or more other identified biomarkers so as to allow correlation to the biological state of interest as defined herein. Specific examples of biomarkers covered by the present invention include kinases, specifically protein kinases, more specifically protein kinase C, more specifically protein kinase C delta and its isozymes such as PKCδI and PKCδVIII.

The term “biological state” as used herein refers to the result of the occurrence of a series of biological processes. As the biological processes change relative to each other, the biological state also changes. One measurement of a biological state is the level of activity of biological variables such as biomarkers, parameters, and/or processes at a specified time or under specified experimental or environmental conditions. A biological state can include, for example, the state of an individual cell, a tissue, an organ, and/or a multicellular organism. A biological state can be measured in samples taken from a normal subject or a diseased subject thus measuring the biological state at different time intervals may indicate the progression of a disease in a subject. The biological state may include a state that is indicative of disease (e.g. diagnosis); a state that is indicative of the progression or regression of the disease (e.g. prognosis); a state that is indicative of the susceptibility (risk) of a subject to the disease; and a state that is indicative of the efficacy of a treatment of the disease.

The term “baseline level” or “control level” of biomarker expression or activity refers to the level against which biomarker expression in the test sample can be compared. In some embodiments, the baseline level can be a normal level, meaning the level in a sample from a normal patient. This allows a determination based on the baseline level of biomarker expression or biological activity, whether a sample to be evaluated for disease cell growth has a measurable increase, decrease, or substantially no change in biomarker expression as compared to the baseline level. The term “negative control” used in reference to a baseline level of biomarker expression generally refers to a baseline level established in a sample from the subject or from a population of individuals which is believed to be normal (e.g. non-tumorous, not undergoing neoplastic transformation, not exhibiting inappropriate cell growth). In other embodiments, the baseline level can be indicative of a positive diagnosis of disease (e.g. positive control). The term “positive control” as used herein refers to a level of biomarker expression or biological activity established in a sample from a subject, from another individual, or from a population of individuals, where the sample was believed, based on data from that sample, to have the disease (e.g. tumorous, cancerous, exhibiting inappropriate cell growth). In other embodiments, the baseline level can be established from a previous sample from the subject being tested, so that the disease progression or regression of the subject can be monitored over time and/or the efficacy of treatment can be evaluated.

The term “cancer”, “tumor”, “cancerous”, and malignant” as used herein, refer to the physiological condition in mammals that is typically characterized by unregulated cell growth. Examples of cancer include, but are not limited to, tumors in neural tissue such as gliomas, neuroblastomas, neuroepitheliomatous tumors, and nerve sheath tumors.

The term “neurodegenerative disease” refers to any abnormal physical or mental behavior or experience where the death or dysfunction of neuronal cells is involved in the etiology of the disorder. Examples of neurodegenerative diseases include, but are not limited to, Alzheimer's disease, Parkinson's disease, Huntington's disease, dementia, amyotrophic lateral sclerosis (ALS), and multiple sclerosis.

The term “about” as used herein is not intended to limit the scope of the invention but instead encompass the specified material, parameter or step as well as those that do not materially affect the basic and novel characteristics of the invention.

The terms “effective amount” for purposes herein is thus determined by such considerations as are known in the art. An effective amount of a compound such as retinoic acid is that amount necessary to provide a therapeutically effective result in vivo or in vitro. The amount of such compound must be effective to achieve a response, including but not limited to increasing or decreasing levels of an isozyme (particularly increasing levels of PKCδVIII), increasing or decreasing levels of a splicing factor (particularly increasing levels of SC35), total prevention of (e.g., protection against) and to improved survival rate or more rapid recovery, or improvement or elimination of symptoms associated with neurological disorders, neurodegenerative diseases, neuronal metastases, etc. or other indicators as are selected as appropriate measures by those skilled in the art. In accordance with the present invention, a suitable single dose size is a dose that is capable of preventing or alleviating (reducing or eliminating) a symptom in a subject when administered one or more times over a suitable time period. One of skill in the art can readily determine appropriate single dose sizes for systemic administration based on the size of a mammal and the route of administration. The terms “effective amount” are used synonymously with the terms “therapeutically effective amount”.

Vitamin A, an important micronutrient and its active metabolite all-trans-retinoic acid (RA) influence a broad range of physiological and pathological processes in the embryonic central nervous system and in the mature brain. Protein kinase C (PKC), a serine/threonine kinase family, consists of 11 isoforms and their splice variants and is involved in the regulation of cellular differentiation, growth, and apoptosis (Nishizuka, Y. (1986) Science 233, 305-312). Protein kinase Cδ, a member of the novel PKC subfamily, is implicated in both apoptosis and cell survival pathways ((Emoto, Y., Manome, Y., Meinhardt, G., Kisaki, H., Kharbanda, S., Robertson, M., Ghayur, T., Wong, W. W., Kamen, R., and Weichselbaum, R. (1995) EMBO J. 14, 6148-6156; Ghayur, T., Hugunin, M., Talanian, R. V., Ratnofsky, S., Quinlan, C., Emoto, Y., Pandey, P., Datta, R., Huang, Y., Kharbanda, S., Allen, H., Kamen, R., Wong, W., and Kufe, D. (1996) J. Exp. Med. 184, 2399-2404; Kohtz, J. D., Jamison, S. F., Will, C. L., Zuo, P., Lu\ddot{}hrmann, R., Barcia-Blanco, M. A., and Manley, J. L. (1994) Nature 368, 119-124; Anantharam, V., Kitazawa, M., Wagner, J., Kaul, S., and Kanthasamy, A. G. (2002) J. Neurosci. 22, 1738-1751; Reyland, M. E., Anderson, S. M., Matassa, A. A., Barzen, K. A., and Quissell, D. O. (1999) J. Biol. Chem. 274, 19115-19123; Denning, M. F., Wang, Y., Tibudan, S., Alkan, S., Nickoloff, B. J., and Qin, J. Z. (2002) Cell Death Differ. 9, 40-52; Sitailo, L., Tibudan, S., and Denning, M. F. (2004) J. Invest. Dermatol. 123, 1-10; Sitailo, L. A., Tibudan, S. S., and Denning, M. F. (2006) J. Biol. Chem. 281, 29703-29710); Peluso, J. J., Pappalardo, A., and Fernandez, G. (2001) Endocrinology 142, 4203-4211; Kilpatrick, L. E., Lee, J. Y., Haines, K. M., Campbell, D. E., Sullivan, K. E., and Korchak, H. M. (2002) Am. J. Physiol. Cell Physiol. 283, C48-C57; Zrachia, A., Dobroslav, M., Blass, M., Kazimirsky, G., Kronfeld, I., Blumberg, P. M., Kobiler, D., Lustig, S., and Brodie, C. (2002) J. Biol. Chem. 277, 23693-23701; McCracken, M. A., Miraglia, L. J., McKay, R. A., and Strobl, J. S. (2003) Mol. Cancer. Ther. 2, 273-281) Thus, PKCδ has dual effects and represents a switch that determines cell survival and fate. This can be explained by the expression of alternatively spliced variants of PKCδ with distinct functions in the apoptotic cascade. The occurrence of PKCδ isoforms is species-specific. PKCδI is ubiquitously present in all species while PKCδII, -δIV, -δV, -δVI, and -δVII isoforms are present in mouse tissues (Sakurai, Y., Onishi, Y., Tanimoto, Y., and Kizaki, H. (2001) Biol. Pharm. Bull. 24, 973-977; Kawaguchi, T., Niino, Y., Ohtaki, H., Kikuyama, S., and Shioda, S. (2006) FEBS Lett. 580, 2458-2464); PKCδIII is present in rats and PKCδVIII is present in humans (Ueyama, T., Ren, Y., Ohmori, S., Sakai, K., Tamaki, N., and Saito, N. (2000) Biochem. Biophys. Res. Commun. 269, 557-563; Jiang, K., Apostolatos, A. H., Ghansah, T., Watson, J. E., Vickers, T., Cooper, D. R., Epling-Burnette, P. K., and Patel, N. A. (2008) Biochemistry 47, 787-797).

An important mechanism of regulating gene expression occurs by alternative splicing which expands the coding capacity of a single gene to produce different proteins with distinct functions. (Hastings, M. L., and Krainer, A. R. (2001) Curr. Opin Cell Biol. 13, 302-309) It is now established that close to 90% of human genes undergo alternative splicing and encode for at least two isoforms. Divergence observed in gene expression because of alternative splicing may be tissue-specific, developmentally regulated or hormonally regulated (Kawahigashi, H., Harada, Y., Asano, A., and Nakamura, M. (1998) Biochim. Biophys. Acta 1397, 305-315; Libri, D., Piseri, A., and Fiszman, M. Y. (1991) Science 252, 1842-1845); Muro, A. F., Iaconcig, A., and Baralle, F. E. (1998) FEBS Lett. 437, 137-141; Du, K., Peng, Y., Greenbaum, L. E., Haber, B. A., and Taub, R. (1997) MCB 17, 4096-4104; Chalfant, C. E., Mischak, H., Watson, J. E., Winkler, B. C., Goodnight, J., Farese, R. V., and Cooper, D. R. (1995) J. Biol. Chem. 270, 13326-13332; Patel, N. A., Chalfant, C. E., Watson, J. E., Wyatt, J. R., Dean, N. M., Eichler, D. C., and Cooper, D. R. (2001) J. Biol. Chem. 276, 22648-22654). Of utmost scientific interest is the study of physiological systems in which the splicing pattern changes in response to a stimulus such as a hormone or a nutrient.

Recently, the inventors identified a new splice variant of human PKCδ, PKCδVIII (GenBank™ Accession No. DQ516383). Sequencing and computational analysis of the PKCδVIII sequence indicated that this human splice variant is generated by utilization of an alternative downstream 5′ splice site of PKCδ pre-mRNA exon 10. (Jiang, K., Apostolatos, A. H., Ghansah, T., Watson, J. E., Vickers, T., Cooper, D. R., Epling-Burnette, P. K., and Patel, N. A. (2008) Biochemistry 47, 787-797) Further, the inventors demonstrated that RA dramatically increased the expression of PKCδVIII via alternative splicing in NT2 cells. RA promotes hippocampal neurogenesis and spatial memory. (Bonnet, E., Touyarot, K., Alfos, S., Pallet, V., Higueret, P., and Abrous, D. N. (2008) PLoS ONE 3, e3487) RA is an early signaling component of the central nervous system (CNS) and acts as a master switch of gene expression. It is well established that the vitamin A metabolite, RA, directly affects transcription of genes. Hence, the inventors sought to elucidate the molecular mechanisms governing this novel observation of RA-mediated alternative splicing of PKCδ pre-mRNA resulting in the expression of the pro-survival protein PKCδVIII.

### EXPERIMENTAL PROCEDURES

Cell Culture

The Ntera2 human teratocarcinoma cell line (NT2/D1 cells) is maintained in DMEM, 10% fetal bovine serum (FBS) with fresh medium about every 3 days. The cells are supplemented with about 10 μM RA as indicated.

Primary Human Neuronal Cells

cDNA from these cells were obtained from Dr. Sanchez-Ramos (James A. Haley Veterans Hospital, Tampa, Fla.), and the cells were cultured in his laboratory. Patients undergoing anterior temporal lobectomy provided written informed consent allowing the tissue to be used for research. The study was approved by the Institutional Review Board (IRB 102342), University of South Florida. Hippocampal tissue was dissected from the temporal lobe resection, dissociated, and plated for generation of a stem/progenitor cells line using standard methods. Hippocampus biopsies were sterilely removed from a 31-year-old male and transferred to a 35-mm plate containing PBS plus 0.5% BSA. A sterile scalpel was used to finely chop the tissue into small pieces. 0.05% Trypsin/EDTA was added to cells and was incubated at about 37° C. for about 8-10 min. The pellet was suspended in DMEM/F12 plus 10% FBS, followed by DNase treatment. The final pellet was re-suspended in DMEM/F12, and a cell count for viability was performed. The cells were seeded into a T-75 flask in DMEM/F12 plus 2% FBS, EGF, and bFGF 20 ng/ml. Cells were replated on poly-L-ornithine-coated chamber slides. Digital images of the hippocampal neurons stained with nestin, TuJ1, BrdU, and NeuN were captured using Zeiss confocal microscope and characterized. The cells were maintained at about 37° C. in about 5% CO₂, about 95% humidity. As the numbers of proliferating cells reached confluency, aliquots of stem/progenitor cells were frozen for later use. Cells used in experiments described here were plated into 6-well plates.

Western Blot Analysis

Cell lysates (about 40 μg) were separated on 10% SDS-PAGE. Proteins were electrophoretically transferred to nitrocellulose membranes, blocked with Tris-buffered saline, 0.1% Tween 20 containing 5% nonfat dried milk, washed, and incubated with a polyclonal antibody against either anti-SC35, anti-SF2/ASF, anti-PKCδ (BioSource), or PKCδVIII specific polyclonal antibody. PKCVIII polyclonal antibody was raised in rabbits by Bio-Synthesis, Inc., Louiseville, Tex. to the synthetic peptide NH2-HISGEAGSIAPLRFLFPLRPKKGDC-COOH (SEQ ID No: 1) (amino acids 329-351, corresponding to the V3-hinge domain of PKCδVIII). The antibody was characterized alongside unreactive pre-immune antisera and will be shown to recognize PKCVIII in samples. This antibody is specific for PKCδVIII as it recognizes the extended hinge region which is absent in PKCδI. (Jiang, K., Apostolatos, A. H., Ghansah, T., Watson, J. E., Vickers, T., Cooper, D. R., Epling-Burnette, P. K., and Patel, N. A. (2008) Biochemistry 47, 787-797; Jiang, K., Patel, N. A., Watson, J. E., Apostolatos, H., Kleiman, E., Hanson, O., Hagiwara, M., and Cooper, D. R. (2009) Endocrinology 150, 2087-2097) Following incubation with anti-rabbit IgG-HRP, enhanced chemiluminescence (Pierce™) was used for detection. In apoptotic cells, PARP is cleaved by caspase 3 into an 85 kDa fragment which is detected in addition to the 116 KDa fragment using anti-PARP antibody in western blot analysis. (PARP) is differentially processed in apoptosis and necrosis and hence its activity can be used as a means of distinguishing the two forms of cell death. (Putt K S, Beilman G J, and H. P J., Direct quantitation of poly(ADP-ribose) polymerase (PARP) activity as a means to distinguish necrotic and apoptotic death in cell and tissue samples. Chembiochem, 2005. 6: p. 53-55)

Quantitative Real-Time RT-PCR:

cDNA (about 2 μl) was amplified by real-time quantitative PCR using Syber (SYBR) Green with an ABI PRISM 7900 sequence detection system (PE Applied Biosystems, Foster City, Calif.) as described previously to quantify absolute levels of PKCδI and PKCδVIII mRNA in the samples (Jiang, K., Apostolatos, A. H., Ghansah, T., Watson, J. E., Vickers, T., Cooper, D. R., Epling-Burnette, P. K., and Patel, N. A. (2008) Biochemistry 47, 787-797). GAPDH was amplified as the endogenous control. Briefly, primers used were as follows:

PKCδI Sense Primer:

5′-GCCAACCTCTGCGGCATCA-3′ (SEQ ID No: 2); antisense primer: 5′-CGTAGGTCCCACTGTTGTC2TTGCATG-3′ SEQ ID No: 3); PKCδVIII sense primer: 5′-GCCAACCTCTGCGGCATCA-3′ (SEQ ID No: 4); antisense primer: 5′-CGTAGGTCCCACTGTTGTC2CTGTCTC-3′ (SEQ ID No: 5). These primers overlap the exon-exon boundary specific for each transcript.

The Primers for GAPDH Were:

sense primer 5′-CTTCATTGACCTCAACTACAT-3′(SEQ ID No: 6) and antisense primer 5′-TGTCATGGATGACCTTGGCCA-3′ (SEQ ID No: 7). Real time PCR was then performed on samples and standards in triplicates. Absolute quantification of mRNA expression levels for PKCδI and PKCδVIII was calculated by normalizing the values to GAPDH. The results were analyzed with two-tailed Student's t test using PRISM4 statistical analysis software (GraphPad, San Diego, Calif.). A level of p<0.05 was considered statistically significant. Significance is determined after three or more experiments.

Transient Transfection of Plasmid DNA:

SC35 and SF2/ASF plasmids were obtained from Origene (TrueClone™ cDNA plasmids). Plasmid DNA (about 1 to about 2 μg) was transfected into cells using Trans-IT®, or Lipofectamine® (Invitrogen) per the manufacturer's instructions.

siRNA Transfection:

Two siRNAs that target separate areas were used to knockdown expression of SC35. SC35 siRNAs along with its scrambled control were purchased from Ambion® (IDs: 12628 and 12444) and transfected using Ambion's siRNA transfection kit. These were validated for specificity to eliminate off-target gene effects. Ambion's PARIS kit (catalogue 1921) was used to simultaneously isolate proteins and RNA to verify knockdown by siRNA transfection.

RT-PCR

Total RNA was isolated from cells using RNA-Bee™ (Tel Test, Inc) as per manufacturer's instructions. About 2 μg of RNA was used to synthesize first strand cDNA using an Oligo(dT) primer and Omniscript™ kit (Qiagen). PCR was performed using about 2 μl of RT reaction and Takara Taq polymerase.

The Primers are Listed:

Human PKCδ sense primer 5′-CACTATATTCCAGAAAGAACGC-3′ (SEQ ID No: 8) and antisense primer 5′-CCCTCCCAGATCTTGCC-3′ (SEQ ID No: 9); PKCδVIII-specific antisense primer 5′-CCCTCCCAGATCTTGCC-3′ (SEQ ID No: 10); SD-SA on pSPL3 sense primer 5′-TCTCAGTCACCTGGACAACC-3′ (SEQ ID No: 11) and antisense primer 5′-CCACACCAGCCACCACCTTCT-3′ (SEQ ID No:12); SC35 sense primer 5′-TCCAAGTCCAAGTCCTCCTC-3′ (SEQ ID No: 13) and antisense primer 5′-ACTGCTCCCTCTTCTTCTGG-3′ (SEQ ID No: 14); GAPDH sense primer 5′-CTTCATTGACCTCAACTCATG-3′ (SEQ ID No: 6) and antisense primer 5′-TGTCATGGATGACCTTGGCCAG-3′ (SEQ ID No: 7).

Using PKCδ primers, PKCδI and PKCδVIII are detected simultaneously: PKCδI is 368 bp and PKCδVIII is 461 bp. Using PKCδVIII-specific primers, PKCδVIII is 424 bp; SC35 is 210 bp; GAPDH is 391 bp; SD-SA: 263 bp; utilization of 5′ splice site I: 419 bp; utilization of 5′ splice site II: 512 bp. About 5% of products were resolved on 6% PAGE gels and detected by silver staining. The PCR reaction was optimized for linear range amplification to allow for quantification of products. Densitometric analyses of bands were done using Un-Scan IT™ Analysis Software (Silk Scientific).

Construction of pSPL3-PKCδ Minigenes:

The pSPL3 vector contains an HIV genomic fragment with truncated tat exons 2 and 3 inserted into rabbit β-globin coding sequences. (Church, D. M., Stotler, C. J., Rutter, J. L., Murrell, J. R., Trofatter, J. A., and Buckler, A. J. (1994) Nat. Genet. 6, 98-105) The resulting hybrid exons in pSPL3 are globin E1E2-tat exon 2 and tat exon 3-globin E3 separated by more than 2.5 kilobase pairs of tat intron sequence. pSPL3 contains a multiple cloning sequence (MCS) around 300 nucleotides downstream of the tat exon 2 5′ splice site. The SV40 promoter and polyadenylation signal allow for enhanced expression in NT2 cells. There are several cryptic 5′ splice sites, which interfere with minigene splicing and hence sections of the original pSPL3 vector were deleted.

First, 874 bp of the tat intronic section lying upstream of SA was deleted. It was designed such that the deletion began 158 bp upstream of SA thereby maintaining the branch point and pyrimidine tract. Primers to amplify genomic PKCδ from NT2 cells were designed using the Gene Tool Software (Bio Tools Inc.) and include the BclI site in the forward primer (in bold type) and BcuI site in the reverse primers (in bold type). The forward primer was designed such that the product will contain the branch point and 3′ splice site. Following amplification of the product, it was ligated into the digested pSPL3 vector. The pSPL3 vector was digested with BamHI (in the MCS) and NheI within the tat intronic sequence which removes an additional 930 bases. The overhangs of the selected restriction enzymes can hybridize and this enabled cloning of the PCR product in the proper orientation. To increase the efficiency and number of positive clones, the ligation reaction was digested with the above restriction enzymes, which cleave any dimers produced by the ligation reaction. The product was verified by restriction digestion and sequencing. The primers used to generate pSPL3-PKCδ minigene were: forward primer 5′ CCTTGATCATGGGAGTTCTGATAATGGTC 3′ (SEQ ID No: 15); reverse primer 5′ CCTACTAGTATCGGGTCTCAGTCTACAC 3′ (SEQ ID No: 16) such that 200 bp of the 5′ intronic sequence was included. The products were ligated into the digested pSPL3 vector and transformed into bacteria using TOP10F cells (Invitrogen). Truncated minigenes were verified by restriction digestion and sequencing.

Site-Directed Mutagenesis

The SC35 cis-element (sequence: ggccaaag) (SEQ ID No: 17) identified on the 5′ intronic sequence flanking exon 10 of PKCδ pre-mRNA was mutated in the pSPL3_PKCδ minigene to tagcccata (SEQ ID No: 18) using QuikChange® site-directed mutagenesis kit (Stratagene), which allows for blue-white screening per the manufacturer's instructions. The mutated minigene, pSPL3_PKCδ**, was verified by sequencing.

RNA Binding Assays

The templates used were F1 (which contains PKCδ exon 10 and 120 bp of its 5′ intronic sequence including the putative SC35 binding site); mutated F1 (F1m, same region as F1 but putative SC35 binding site was mutated as described above) and F2 (which is PKCδ exon 10 alone). Single-stranded RNAs were synthesized in vitro using the T7 RNA polymerase and purified on denaturing polyacrylamide gels prior to RNA binding assays. The transcripts were 5′ biotinylated with about 0.1 mM biotin-21 as described previously. (Gallego, M. E., Gattoni, R., Step'venin, J., Marie, J., and Expert-Bezanc, on, A. (1997) EMBO J. 16, 1772-1784) RNA gel shift mobility assay was performed with about 10 fmol of labeled RNA and about 5 ng of recombinant SC35 (ProteinOne) in about a 20-μl binding reaction (about 100 mM Tris, about 500 mM KCl, about 10 mM dithiothreitol, about 2.5% glycerol, about 2 units/μl RNAsin) and incubated at about 30° C. for about 20 min. The complex was run on 8% polyacrylamide gel and transferred to a nylon membrane. Western blot analysis was performed using avidin-HRP conjugate (Pierce).

Statistical Analysis

Gels were densitometrically analyzed using UN-SCAN-IT™ software (Silk Scientific, Inc.) PRISM™ software was used for statistical analysis. The results were expressed as mean±S.E. of densitometric units or as percent exon inclusion.

Expression of PKCδVIII

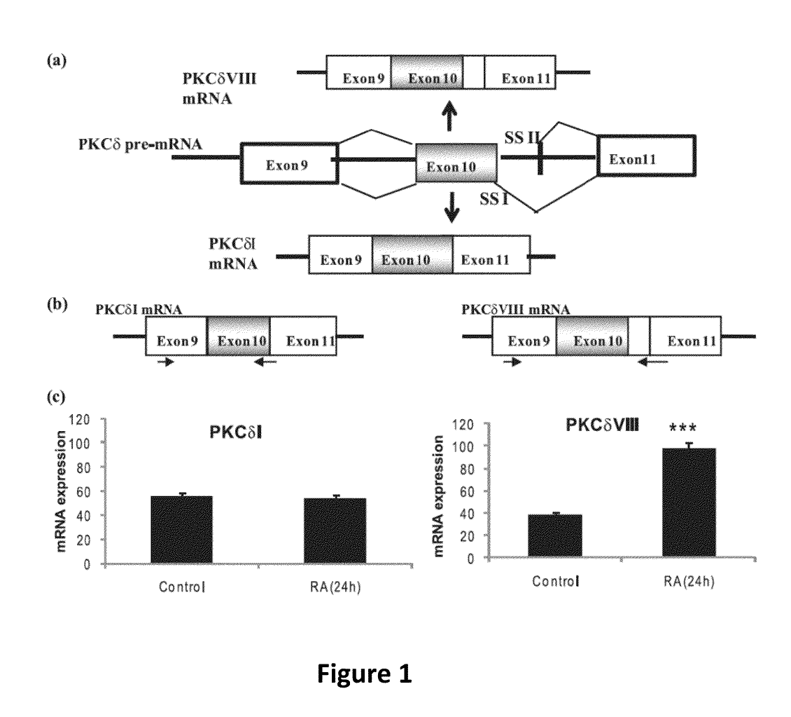

In humans, the PKCδ gene has at least two alternatively spliced variants: PKCδI and PKCδVIII (FIG. 1a). Human PKCδI mRNA sequence coding for 674 amino acids has a molecular mass of 78 kDa while PKCδVIII mRNA sequence codes for 705 amino acids and has a molecular mass of ˜81 kDa. Retinoic acid regulates the expression of the human splice variant PKCδVIII, generated by utilization of an alternative downstream 5′ splice site of PKCδ pre-mRNA exon 10 as shown in FIG. 1. PKCδVIII is generated via alternative splicing of the PKCδ pre-mRNA such that 93 nucleotides are included in the mature PKCδVIII mRNA. This translates to 31 amino acids whose inclusion disrupts the caspase-3 recognition sequence in the hinge region of PKCδVIII protein. The inventors have demonstrated that PKCδVIII functions as a pro-survival protein whereas PKCδI promotes apoptosis. Over-expression of PKCδVIII decreases cellular apoptosis and siRNA mediated knockdown of PKCδVIII further demonstrated that PKCδVIII functions as an antiapoptotic protein in NT2 cells. Increased expression of PKCδVIII shields cells from etoposide-mediated apoptosis. Further, RA (about 24 h) significantly increases the expression of PKCδVIII in NT2 cells (Jiang, K., Apostolatos, A. H., Ghansah, T., Watson, J. E., Vickers, T., Cooper, D. R., Epling-Burnette, P. K., and Patel, N. A. (2008) Biochemistry 47, 787-797).

The inventors demonstrate the physiological significance of the expression pattern of PKCδVIII in human hippocampus and its response to RA. The inventors performed quantitative, two-step real-time RT-PCR using Syber (SYBR) Green technology. The primers were specific to the exon junctions of PKCδI mRNA and PKCδVIII mRNA as shown in FIG. 1b. Each transcript was normalized to the endogenous control, GAPDH, to obtain absolute quantification. It was found that PKCδVIII increased with RA treatment whereas PKCδI levels remain constant in human primary neuronal cells (FIG. 1c)

PKCδVIII Expression is Found in the Brain

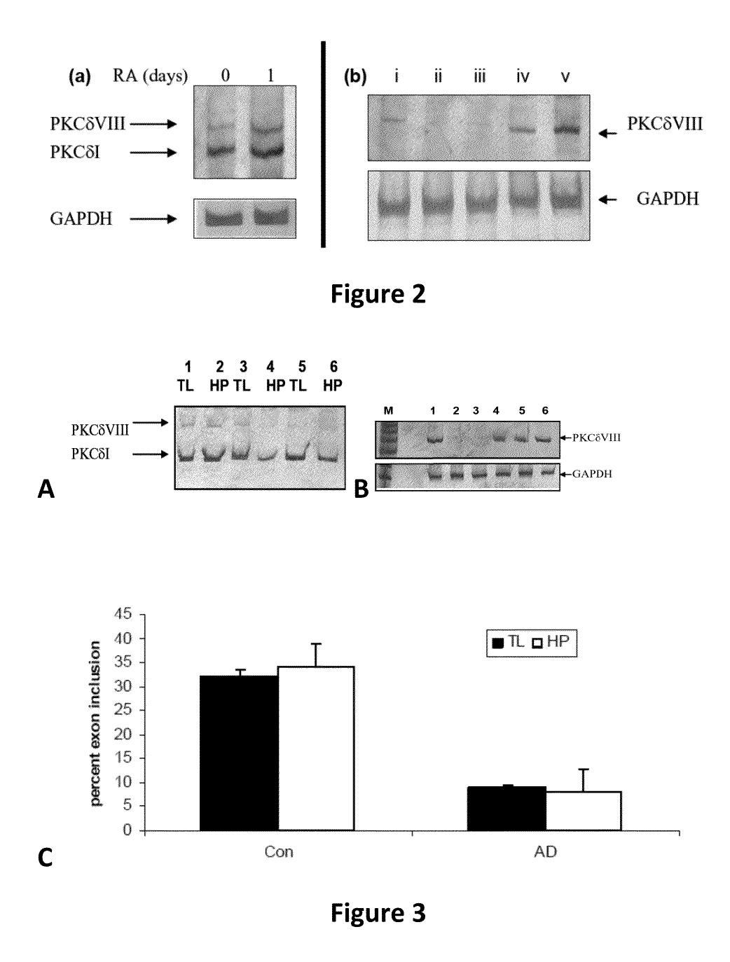

The inventors looked for the expression of PKCδ isozymes in primary neuronal cells to verify the expression pattern of PKCδVIII. A primary human neural cell line was created from adult hippocampus biopsies and these cells were obtained from Dr. Sanchez-Ramos (James A. Haley Veterans Hospital, Tampa, Fla.). Patients undergoing anterior temporal lobectomy for intractable seizures provided informed consent allowing the tissue to be used for research. Hippocampal tissue was dissected from the temporal lobe resection, dissociated and plated for generation of a stem/progenitor cells line using standard methods. As the numbers of proliferating cells reached confluency, aliquots of stem/progenitor cells were frozen for later use. For each experiment, cells were thawed and replated in “proliferation” media. Cells were treated with RA for about 24 h. Total RNA was isolated and RT-PCR was performed with human PKCδ primers which amplify both PKCδI and PKCδVIII products simultaneously. PKCδI and PKCδVIII isoforms were detected and the levels of PKCδVIII increased with retinoic acid treatment (FIG. 2a). PKCδVIII was not detected in aorta smooth muscle cells or skeletal muscle cells (data not shown). Next, human fetal tissue-specific cDNAs (from Origene) were used in the PCR reaction to detect PKCδ isoforms. The expression of PKCδVIII is tissue specific with highest levels seen in the fetal brain (FIG. 2b) compared to other tissues tested (fetal testis, kidney, heart and spleen).

PKCδVIII Expression is Decreased in Alzheimer's Brain Tissues

Temporal lobe and hippocampus are affected early in Alzheimer's disease (AD). The inventors performed RT-PCR analysis using PKCδ primers on samples from AD patient brain (cDNA obtained from Dr. Schellenberg, Va. Medical Center, Seattle). The results showed that PKCδVIII expression is decreased in AD brain (sections: TL: temporal lobe and HP: hippocampus) compared to matched control samples (FIG. 3). This data is representative of about 30 samples analyzed to determine if RNA measurements could be made from human autopsy samples. As shown in FIG. 3, PKCδVIII expression is dramatically decreased in Alzheimer's disease patients compared to their matched controls while increased PKCδVIII levels are observed in glioma and neuroblastoma cell lines (FIGS. 3a, b). These results led the inventors to the conclusion that PKCδVIII expression in neuronal cells could be used as a biomarker for neurodegenerative diseases as well as neuronal cancers.

RA Promotes the Expression of Anti-Apoptotic Proteins Concurrently with Increased Expression of PKCδVIII and Concurrent Expression of Bcl-2.

Recent research has indicated that the adult brain, too, is capable of differentiating and developing neurons. The differentiation and development of neurons in neurogenesis, regeneration and repair is regulated by a fine balance between the pro-apoptotic and anti-apoptotic signals. Various studies involving basic research and stem cells demonstrate the importance of apoptotic balance in the nervous system. (Arvanitakis, Z., et al., Diabetes mellitus and risk of Alzheimer disease and decline in cognitive function. Arch Neurol, 2004. 61(5): p. 661-6; Citron, M., Strategies for disease modification in Alzheimer's disease. Nat Rev Neurosci, 2004. 5(9): p. 677-85; Mattson, M., Pathways towards and away from Alzheimer's disease. Nature, 2004. 430: p. 631-639) Bcl-2 and Bcl-xL, the pro-survival proteins enhance neurogenesis and decrease apoptosis in the brain.

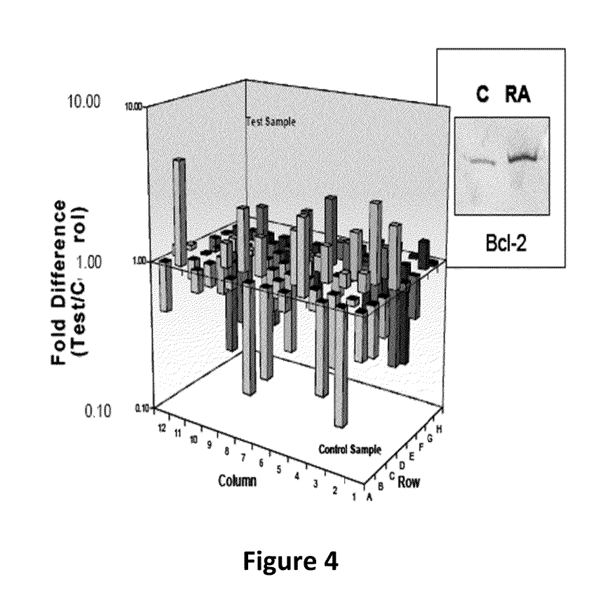

The inventors have shown that retinoic acid increases the levels of PKCδVIII in NT2 cells. An apoptosis micro-array (SuperArray, catalog #PAHS-012A) was used to determine the profiles of proteins associated with the apoptotic cascade. RNA was isolated from control and RA (about 24 h) treated NT2 cells and used in the analysis. Real-time RT-PCR was performed according to the manufacturers' protocol and data was analyzed by SuperArray software (FIG. 4). The inventors observed about a 6-fold increase in Bcl-2 levels which were concurrent with an increase in PKCδVIII levels following RA treatment. Moderate increases in Mcl-1 and A1 were also observed. The inset of FIG. 4 shows the results of PCR using Bcl-2 primers performed on control and RA-treated samples used in the microarray analysis.

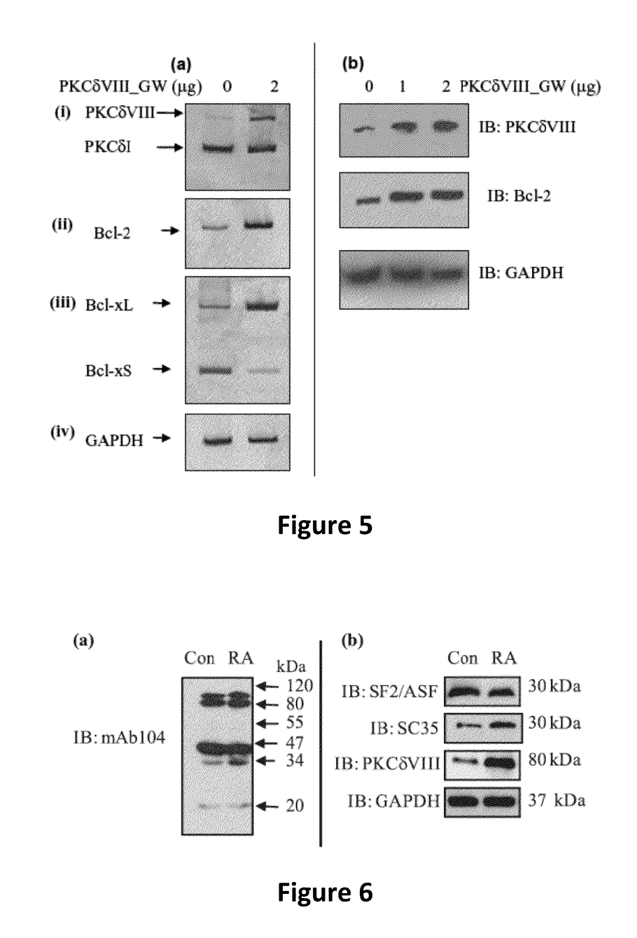

The inventors found that PKCδVIII promotes the expression of Bcl-2 and the increase in Bcl-2 observed above was due to PKCδVIII expression. PKCδVIII cDNA was cloned into the pcDNA™ 6.2/V5 Gateway directional TOPO vector. The expression vector is hereby referred to as PKCδVIII_GW. PKCδVIII_GW was transiently transfected in NT2 cells. Total RNA was isolated and RT-PCR performed using primers for human PKCδ and Bcl-2. Using RT-PCR analysis the inventors observed an increase in the expression of Bcl-2 concomitant with an increase in PKCδVIII expression (FIG. 5a, panels i, ii) thus confirming the results of the micro-array. In separate experiments, PKCδVIII was transfected in increasing amounts and western blot analysis carried out using antibodies against PKCδVIII and Bcl-2 (FIG. 5b). These results confirmed that PKCδVIII promoted the expression of Bcl-2.

PKCδVIII Over-Expression Increases Bcl-xL Levels

The splice variants of Bcl-x are involved in determining the apoptotic fate of neuronal cells. The Bcl-xL isoform promotes survival of cells. The inventors established that PKCδVIII affects the levels of the Bcl-x isoforms. PKCδVIII_GW was transiently transfected in NT2 cells in increasing amounts. Total RNA was isolated and RT-PCR was carried out using primers for Bcl-x such that both the long form (Bcl-xL: pro-survival) and the short form (Bcl-xS: pro-apoptotic) can be detected simultaneously. PKCδVIII increased the expression of Bcl-xL isoform (FIG. 5a, panels i, iii) and decreased Bcl-xS expression. RA-mediated expression of PKCδVIII increases Bcl-2 and Bcl-xL protein levels which are required for the ability of the kinase to inhibit induction of apoptosis. PKCδVIII promotes cell survival via increasing the expression of the anti-apoptotic proteins: Bcl-2 and Bcl-xL.

Concurrent Increases in SC35 and PKCδVIII Levels in RA-mediated PKCδ Alternative Splicing

Alternative splicing is regulated by recruiting trans-factors such as serine-arginine rich

(SR) proteins that bind to exonic or intronic splicing enhancers (ESE, ISE) on the pre-mRNA. Hence, the elucidation of trans-factors involved in RA-mediated PKCδ alternative splicing is of critical importance. NT2 cells were treated with or without RA (about 24 h), and whole cell lysates were analyzed by Western blot analysis using mAb104 antibody that simultaneously detects the phosphoepitopes on all SR proteins. The results indicated that upon RA treatment, SR protein at ˜30 kDa increased in expression (FIG. 6a). SF2/ASF or SC35 (i.e. SRp30a or SRp30b, respectively) are two SR proteins with molecular masses of ˜30 kDa. Hence, antibodies specific to these individual SR proteins were used next. An increase in SC35 (SRp30b) was observed concurrent with increased PKCδVIII levels in response to RA while SF2/ASF (SRp30a) expression remained relatively constant (FIG. 6b). The observed increase of SC35 with RA reflects total expression levels of SC35. The increases seen with mAb104 antibody, which detects the phosphoepitope, is a reflection of its increased expression rather than increased phosphorylation. SC35, also known as SFRS2 or SRp30b, is a member of the SR splicing protein family and functions as a splicing enhancer (Liu, H. X., Chew, S. L., Cartegni, L., Zhang, M. Q., and Krainer, A. R. (2000) Mol. Cell. Biol. 20, 1063-1071).

SC35 Mimics RA-Mediated PKCδVIII Alternative Splicing

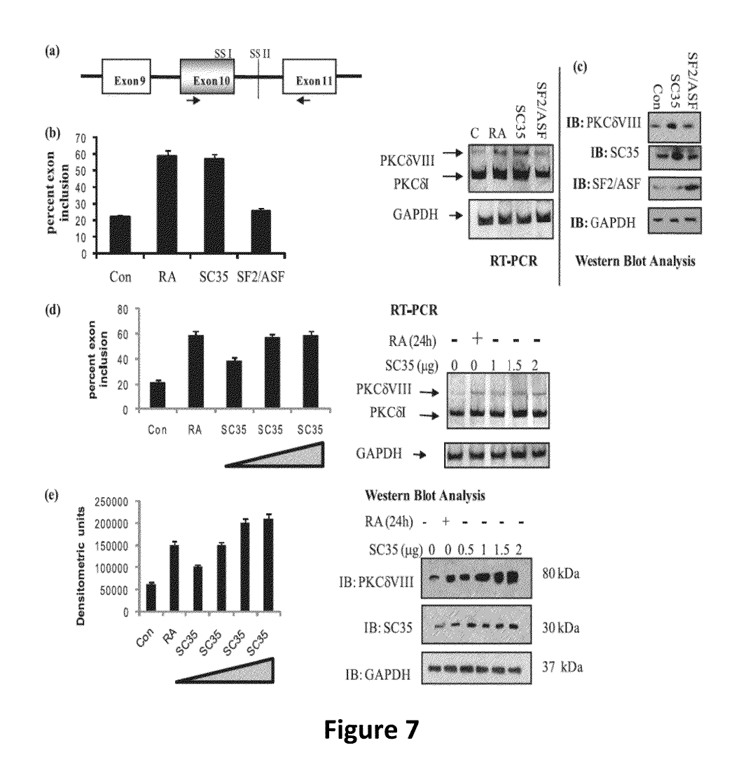

SC35 was transiently transfected into NT2 cells to determine whether it could mimic the effect of RA in increasing the expression of PKCδVIII. SF2/ASF was used as a control and transfected into a separate well. RT-PCR performed using human PKCδ primers which amplified both PKCδI and PKCδVIII products. Simultaneously, Western blot analysis was performed with PKCδVIII-specific antibody. An increase in endogenous PKCδVIII levels in cells overexpressing SC35 was observed (FIG. 7, a-c) while in SF2/ASF transfected cells PKCδVIII expression remained constant. GAPDH was used as internal control for all samples. To determine whether PKCδVIII expression levels increased in direct proportion with SC35, increasing amounts of SC35 (about 0-about 2 μg) were transfected into NT2 cells. Total RNA or whole cell lysates were collected. RT-PCR was performed using PKCδ primers that detect PKCδI and PKCδVIII mRNA, and Western blot analysis was carried out using antibodies for PKCδIII, SC35, and GAPDH (internal control). As seen in FIG. 7d, PKCδVIII mRNA levels increased with increasing levels of SC35 while PKCδI mRNA levels appeared unaffected. Further, PKCδVIII protein levels (FIG. 7e) increased with increasing doses of SC35 comparable to the increase in PKCδVIII protein seen with RA treatment.

RA is Unable to Increase Expression of PKCδVIII in the Absence of SC35

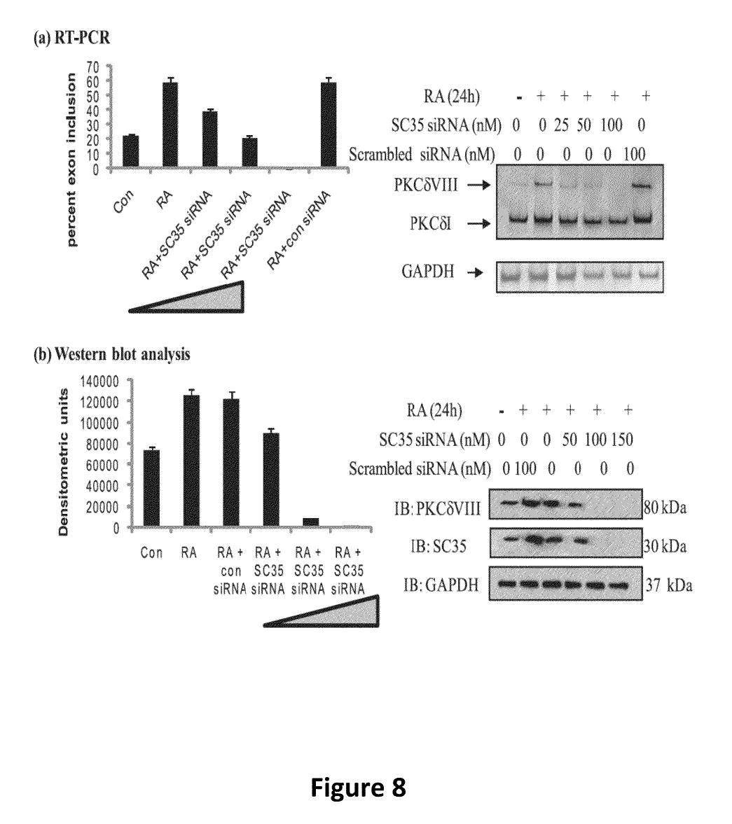

To determine the effect of SC35 knockdown on the RA-mediated expression of PKCδVIII, siRNA specific for SC35 were transfected in increasing amounts (about 0-about 150 nM) into NT2 cells and treated with RA. Two sets of SC35 siRNA along with its scrambled control were used to validate specificity and eliminate off-target knockdowns. Results indicated similar data with either SC35 siRNAs. Total RNA or whole cell lysates were collected. RT-PCR was performed using PKCδ primers while Western blot analysis was carried out using antibodies for PKCδIII, SC35, and GAPDH. As seen in FIG. 8a, PKCδVIII mRNA levels decreased with increasing levels of SC35 siRNA while PKCδI mRNA levels appeared unaffected. Further, PKCδVIII protein levels decreased with increasing doses of SC35 siRNA (FIG. 8b). The graph is representative of four individual experiments performed with either SC35 siRNA. The above data confirms that RA cannot promote PKCδVIII expression in the absence of SC35. This demonstrates the involvement of SC35 in RA-mediated alternative splicing of PKCδ pre-mRNA.

Antisense Oligonucleotides Indicate a Role of SC35 cis-Element in PKCδ Alternative Splicing

Previous studies identified consensus sequences (Ladd, A. N., and Cooper, T. A. (2002) Genomic Biol. 3, 1-16) for several cis-elements present either in the exonic or intronic sequences of pre-mRNA. These consensus sequences serve as a guideline to dissect and analyze putative cis-elements in alternative splicing of pre-mRNA. The inventors combined a web-based resource “ESE finder” (Cartegni, L., Wang, J., Zhu, Z., Zhang, M. Q., and Krainer, A. R. (2003) Nucleic Acids Res. 31, 3568-3571) and also manually checked for published consensus sequences of cis elements on PKCδ pre-mRNA to predict putative enhancer and silencer elements that could recruit trans-factors in RA-regulated alternative splicing of PKCδ. To focus on identifying the cis-elements involved in RA-mediated increase in PKCδVIII mRNA levels, antisense oligonucleotides (ASO) (synthesized by Isis Pharmaceuticals, Carlsbad, Calif.), which are 2′-methoxyethyl-modified, RNase-H resistant were used. These ASOs inhibit binding of trans-factors to their cis-elements without disrupting the splicing event or degrading the mRNA (Patel, N. A., Eichler, D. C., Chappell, D. S., Illingworth, P. A., Chalfant, C. E., Yamamoto, M., Dean, N. M., Wyatt, J. R., Mebert, K., Watson, J. E., and Cooper, D. R. (2003) J. Biol. Chem. 278, 1149-1157; Vickers, T. A., Zhang, H., Graham, M. J., Lemonidis, K. M., Zhao, C., and Dean, N. M. (2006) J. Immunol. 176, 3652-3661).

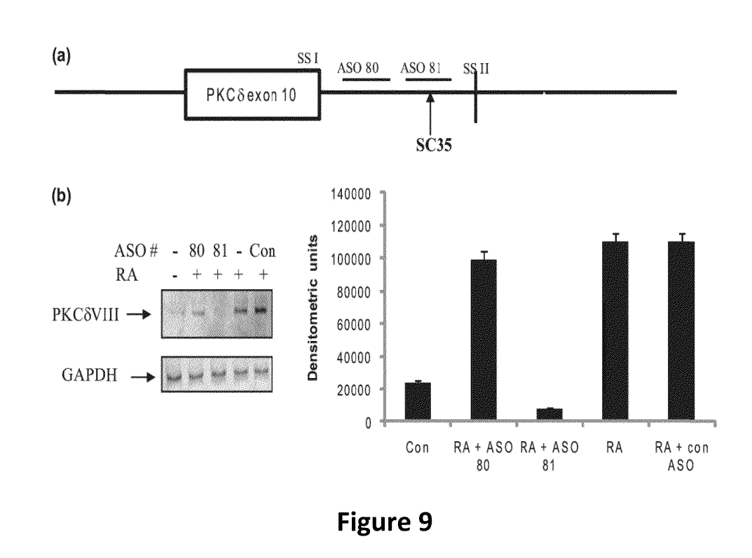

The inventors transfected a series of 20mer ASOs, which were designed according to predicted enhancer and silencer sites such that they sequentially spanned the unspliced PKCδ pre-mRNA. All wells were also treated with RA and RT-PCR was performed. Transfection of ASO 81 (which spans the putative SC35 binding site) showed a significant decrease in RA-induced PKCδVIII splicing while the other ASOs did not affect the expression of PKCδVIII induced by RA (data not shown). Results (FIG. 9, a and b) shown here represent three experiments performed individually using the scrambled ASO as control, ASO 81 and ASO 80 (which was in close proximity to ASO 81 but did not inhibit RA-mediated PKCδVIII alternative splicing). ASO 81 corresponded to the SC35 binding site as identified by ESE finder and further determined by its consensus sequence, ggccaaag. These results demonstrated that ASO 81 inhibited RA induced PKCδVIII alternative splicing. This also suggested the position of SC35 cis-element on PKCδ pre-mRNA to be in the intronic region downstream of PKCδ exon 10 and before 5′ splice site II (schematic in FIG. 9a).

Construction of a Heterologous pSPL3_PKCδ-Splicing Minigene that is Responsive to RA

Preliminary studies found that RARα, β and γ and RXRα were expressed in NT2 cells but not RXRβ nor RXRγ. The biological responses attributed to RA are initiated by binding of the retinoids to its specific receptors (RAR/RXR) in the nucleus of the target cells. The resulting complex binds to the RA-responsive element (RARE) in the promoters of RA-inducible genes. RA mediates its effects through its nuclear receptors RAR/RXR. RARα, β and γ and RXRα were expressed in NT2 cells but not RXRβ nor RXRγ.

It was also found that the PKCδ promoter is responsive to RA. Computational analysis of PKCδ promoter indicated putative RAREs. pGlow-PKCδ promoter (gift from Dr. Stuart H. Yuspa, NCI) was transfected into NT2 cells to determine if RA regulates transcription of the PKCδ gene via RARE on the PKCδ promoter region. RA treatment induced a four-fold increase in fluorescence compared to control samples. This was verified by western blot analysis using GFP antibody to confirm up-regulation of PKCδ promoter by ATRA treatment.

NT2 lysates treated with RA for 0 (control), 1 or 2 days using RNA polymerase II (Covance, 8WG16 which recognizes the C-terminal domain of RNA pol II) were immunoprecipitated to determine whether RNA polymerase II can associate with RXRα or RARs α, β or −γ. Anti-RXRα, anti-RARα, anti-RARβ, or anti-RARγ were then used to immunoblot. It was found that RXRα and RARα associated with RNA polymerase II. RNA polymerase II has also been shown to associate with SC35 as well as with RAREs in response to ATRA using ChIP assays. Taking this data along with the fact that ATRA induces alternative splicing of PKCδ with the involvement of SC35, it was found that SC35 is recruited by RNA polymerase II complex to promote PKCδ splicing in NT2 cells.

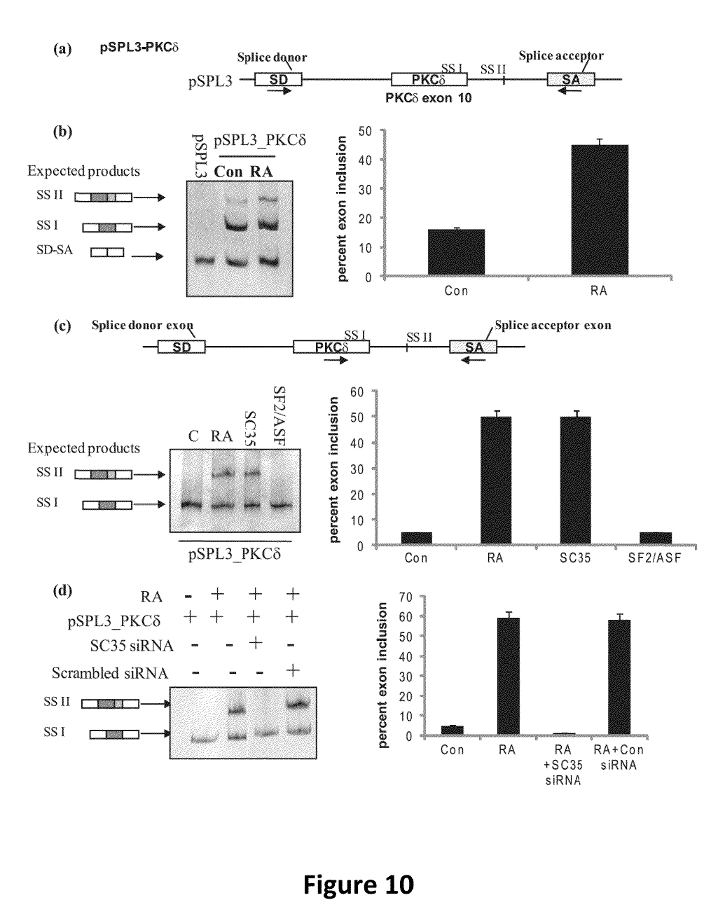

Splicing minigenes are advantageous to identify cis-elements on the pre-mRNA involved in regulated alternative splicing. Further, minigenes aid to correlate the binding of specific SR proteins to individual splicing events. Hence, to dissect the mechanism of RA-mediated regulation of endogenous PKCδ alternative splicing and analyze factors influencing 5′ splice site selection, a PKCδ heterologous minigene was developed. Since the human PKCδ splice variants used alternative 5′ splice sites as determined previously, exon 10 of PKCδ pre-mRNA along with its flanking 3′ and 5′ intronic sequences was cloned (as described under “Experimental Procedures”) in the multiple cloning site (MCS) between the splice donor (SD) and splice acceptor (SA) exons of pSPL3, a vector developed to study splicing events (schematic shown in FIG. 10a). 5′ splice site II (which encodes for PKCδVIII mRNA) is 93 bp downstream of PKCδ exon 10, thus a 200 bp of the 5′ intronic sequence was cloned. The minigene also contains a retinoic acid response element (RARE) in its promoter region. The resulting minigene, pSPL3_PKCδ, was confirmed using restriction digestion and sequencing.

Minigene pSPL3_PKCδ was transfected into NT2 cells; cells were treated with RA (24 h) and RT-PCR performed on total RNA using SD-SA primers. The empty vector pSPL3 with the same modifications used for cloning the minigene, was transfected simultaneously in a separate well. Deletion of intronic sequences between 5′ splice site II and SA exon did not affect RA-mediated utilization of the 5′ splice site II (data not shown) thereby indicating that additional downstream cis-elements were not influencing splice site selection. The predicted products using SD-SA primers are shown (FIG. 10, a and b). RA increased utilization of 5′ splice site II of PKCδ exon 10 in pSPL3_PKCδ minigene thereby mimicking RA mediated increase in endogenous PKCδVIII expression.

Next, the inventors sought to determine if SC35 could increase the utilization of 5′ splice site II on pSPL3_PKCδ minigene such that it mimics the increase of RA-mediated endogenous expression of PKCδVIII. SC35 or SF2/ASF expression vector (2 μg) was co-transfected along with the pSPL3_PKCδ minigene into NT2 cells. RA was added to a separate well transfected with pSPL3_PKCδ minigene. RT-PCR was performed on total RNA using PKCδ exon 10 (sense) and SA (antisense) primers as shown (FIG. 10c). SC35 promoted the selection of 5′ splice site II on PKCδ exon 10 in pSPL3_PKCδ splicing minigene thereby mimicking endogenous RA-mediated increased expression of PKCδVIII.

To show that SC35 is crucial for RA-mediated PKCδVIII 5′ splice site selection, SC35 siRNA was co-transfected with pSPL3_PKCδ minigene in NT2 cells. RA was added to the cells as indicated in the figure. RT-PCR was performed on total RNA using PKCδ exon 10 (sense) and SA (antisense) primers (FIG. 10d). RA treatment could not promote utilization of PKCδVIII 5′ splice site II when SC35 was knocked down. This verified that SC35 was a crucial trans-factor involved in RA-mediated PKCδVIII expression.

Mutation of SC35 Binding Site on the Heterologous pSPL3-PKCδ Minigene Disrupted Utilization of 5′ Splice Site II

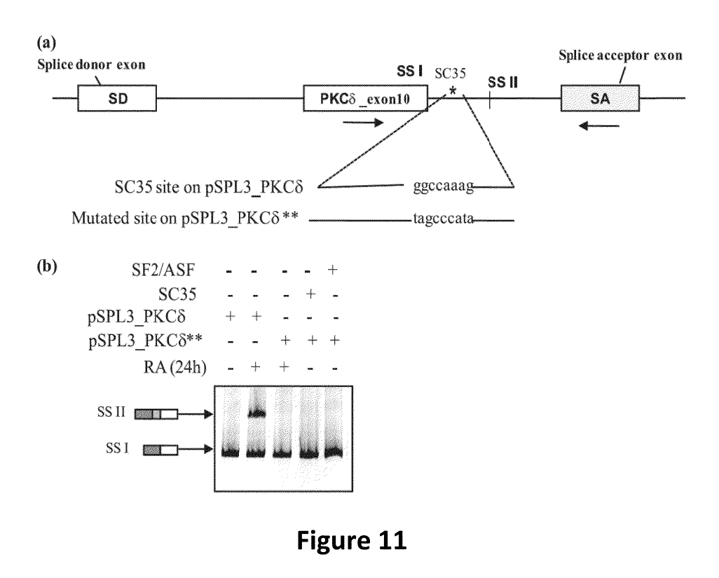

The putative SC35 site identified by its consensus sequence and ASO binding assay (FIG. 9, a and b, above) is in the intronic region between 5′ splice site 1 and 5′ splice site II of PKCδ exon 10. To establish that the putative sequence was an SC35 cis element and that it is essential for RA-mediated PKCδVIII alternative splicing, the intronic SC35 cis-element “ggccaaag” (SEQ ID No: 17) was mutated (FIG. 11a). This site was mutated to “tagcccata” (SEQ ID No: 18) within the pSPL3_PKCδ minigene (described under “Experimental Procedures”) and the mutated pSPL3_PKCδ** minigene was transfected into NT2 cells. The original pSPL3_PKCδ minigene was transfected into a separate well as the control. RA was added for about 24 h as indicated in the figure. In separate wells, SC35 or SF2/ASF was transfected along with the mutated pSPL3_PKCδ** minigene, treated with or without RA. RT-PCR was performed on total RNA using PKCδ exon 10 (sense) and SA (antisense) primers. RA treatment or overexpression of SC35 did not promote the selection of 5′ splice site II on PKCδ exon 10 in the pSPL3_PKCδ**-mutated minigene (FIG. 11b). This experiment demonstrates that the mutated minigene was insensitive to RA treatment and SC35 levels. Further, this indicated that the sequence ggccaaag on PKCδ pre-mRNA was required for RA-mediated PKCδVIII alternative splicing and was a putative binding site for SC35 which is essential for an RA response in PKCδ pre-mRNA 5′ splice site II selection.

SC35 Binds to the Cis-Element on PKCδ Pre-mRNA

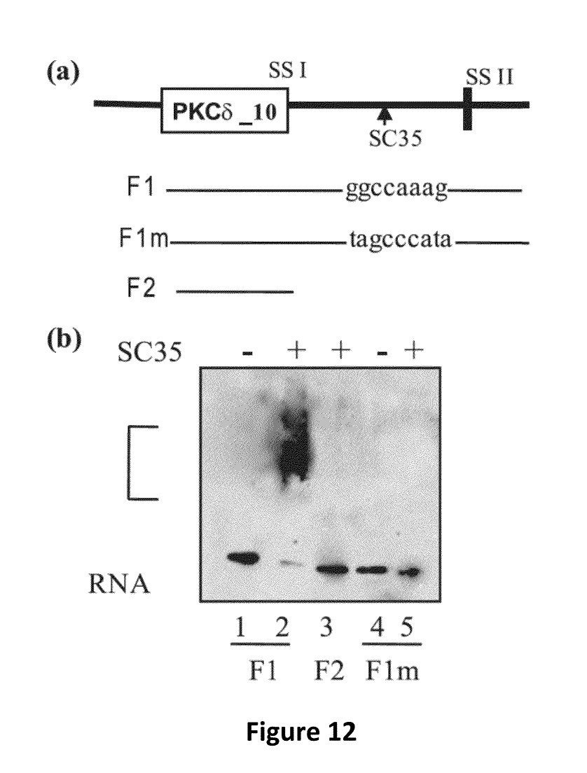

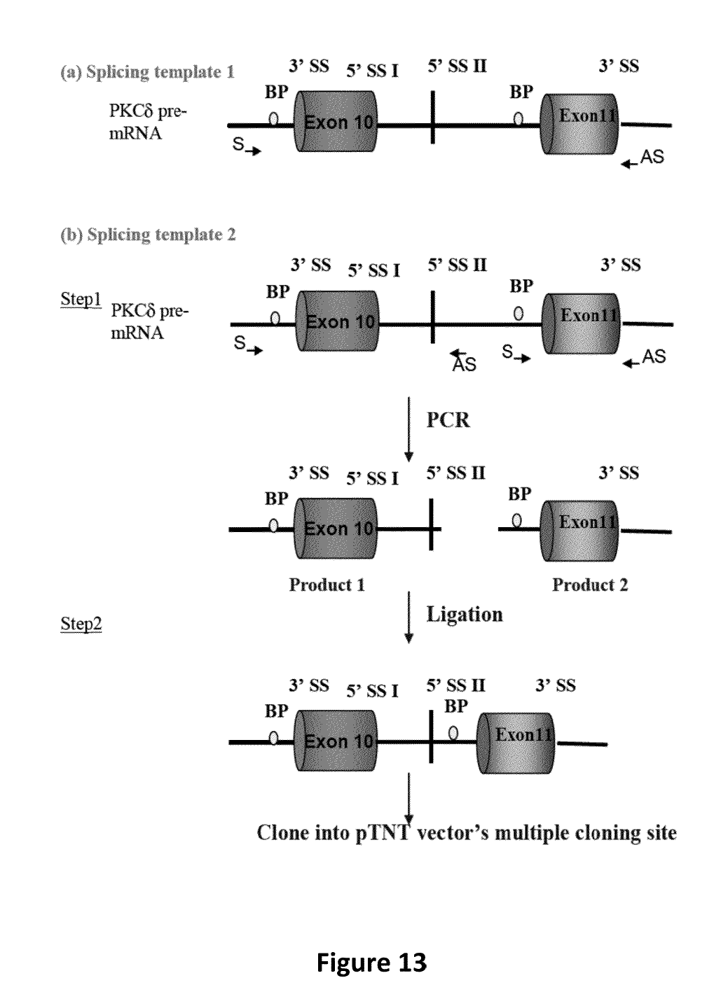

The above experiments demonstrated that SC35 is required for RA mediated increased utilization of 5′ splice site II on PKCδ pre-mRNA, and the enhancer element “ggccaaag” (SEQ ID No: 17) is required for SC35-mediated utilization of 5′ splice site II and hence PKCδVIII alternative splicing. Hence, it was necessary to determine whether this cis-element is a SC35 binding site by performing RNA gel shift assays. Biotin-labeled RNA fragments were synthesized in vitro and tested for interaction with purified recombinant SC35. The RNA transcript F1 contained PKCδ exon 10 and 120 by of its flanking 5′ region, which included the putative SC35 cis-element. The RNA transcript F1m has the putative SC35 binding site mutated as described above. RNA transcript F2 contained only the PKCδ exon 10. As shown in FIG. 12, a and b, F2 did not show any gel shift with SC35 indicating that this transcript did not contain a SC35 binding site. There is a gel shift observed with F1 and SC35 indicating that it contains the SC35 binding site and the recombinant SC35 is able to bind to the RNA. There is no binding observed with F1m and SC35 indicating that the SC35 binding site was abolished. These experiments demonstrate that the enhancer element ggccaaag present in the 5′ region of PKCδ exon 10 pre-mRNA is a SC35 cis-element.

The inventors have shown that the splicing factor SC35 plays an important role in RA-mediated alternative splicing of PKCδVIII pre-mRNA. Alternative pre-mRNA splicing generates protein diversity such that humans express more than 100,000 proteins from only about 25,000 protein coding genes. Defective alternative splicing causes a large number of diseases (D'Souza, I., and Schellenberg, G. D. (2005) Biochim. Biophys. Acta 1739, 104-115 38. Khoo, B., Akker, S. A., and Chew, S. L. (2003) Trends Biotechnol. 21, 328-330; Stamm, S. (2002) Hum. Mol. Genet. 11, 2409-2416). Alternative splicing occurs through various mechanisms such as exon skipping, exon inclusion, alternative 3′ splice site usage, alternative 5′ splice site usage, or alternative polyadenylation site usage. The spliceosome catalyzes the pre-mRNA splicing reaction within a large multicomponent ribonucleoprotein complex comprising of small nuclear RNAs (snRNAs) and associated proteins (such as SR proteins).

Exonic or intronic splicing enhancers (ESE, ISE) in the pre-mRNA bind the serine-arginine-rich nuclear factors (SR proteins) to promote the choice of splice sites. Elucidation of the trans-factors involved in regulated alternative splicing is of critical importance because specific cellular stimuli can favor the binding of certain trans-factors over others, thereby changing the splicing pattern. SC35, also known as SFRS2 or SRp30b, is a splicing enhancer and a member of the SR splicing protein family. It was found that SC35 binds to its cis element on PKCδ pre-mRNA. SC35 has an N-terminal RNA recognition motif (RRM) domain and a C-terminal arginine/serine-rich (RS) domain. The RRM domain interacts and binds to the target pre-mRNA while the RS domain is highly phosphorylated and is the protein interaction region. SC35 also mediates alternative splicing of CD45, tau exon 10 in Alzheimer disease, and neuronal acetylcholinesterase ((Wang, H. Y., Xu, X., Ding, J. H., Bermingham, J. R., Jr., and Fu, X. D. (2001) Mol. Cell. 7, 331-342; Herna'ndez, F., Pe'rez, M., Lucas, J. J., Mata, A. M., Bhat, R., and Avila, J. (2004) J. Biol. Chem. 279, 3801-3806; Meshorer, E., Bryk, B., Toiber, D., Cohen, J., Podoly, E., Dori, A., and Soreq, H. (2005) Mol. Psychiatry. 10, 985-997).

The data demonstrate that SC35 enhances the splicing of a pro-survival protein, PKCδVIII in neurons supporting a role on neurogenesis. The data indicated that the expression levels of SC35 changed with RA treatment rather than significant changes to its phosphorylation (FIG. 6, a and b). Further, inhibitors of several signaling pathways such as PI3K, JAK/STAT, MAPK did not affect RA-mediated PKCδ splicing (data not shown).

The data with primary human neuronal cells demonstrated the physiological significance of the expression pattern of PKCδVIII in human hippocampus and its response to RA. NT2 cells are predominantly used to study neurogenesis, neuronal differentiation, and early development of the nervous system as they represent a culture model for differentiating neurons as well as a potentially important source of cells to treat neurodegenerative diseases (Misiuta, I. E., Anderson, L., McGrogan, M. P., Sanberg, P. R., Willing, A. E., and Zigova, T. (2003) Dev. Brain Res. 145, 107-115). Experiments conducted herein are within the time frame (about 0-about 24 h post-RA) in which NT2 differentiation is compared with normal differentiation in the CNS. This mirrors the time frame in which RA regulates the development of CNS and promotes adult neurogenesis. Because these studies required extensive experiment manipulations and repetitions, they were conducted in human NT2 cells.

Vitamin A and its metabolite, RA, have multiple therapeutic targets and neuroprotective properties. RA regulates neural development as well as its plasticity and promotes early stages of neurogenesis and increases survival. (McCaffery, P., Zhang, J., and Crandall, J. E. (2006) J. NeuroBiol. 66, 780-791) RA also changes the splicing pattern of other genes such as coactivator activator (CoAA) and delta isoform of CaM kinase in P19 embryonal carcinoma stem cells. (Yang, Z., Sui, Y., Xiong, S., Liour, S. S., Phillips, A. C., and Ko, L. (2007) Nucleic Acids Res. 35, 1919-1932; Donai, H., Murakami, T., Amano, T., Sogawa, Y., and Yamaguchi, T. (2000) Brain Res. Mol. Brain. Res. 85, 189-199) However, the mechanism of RA induced splicing of genes had not yet been elucidated. The inventors demonstrate here that the splicing factor, SC35 plays a crucial role in mediating RA effects on alternative splicing of PKCδVIII mRNA in neurons. Understanding how RA regulates gene expression thereby increasing the expression of the pro-survival protein PKCδVIII is a step closer to realizing the therapeutic potential of RA in neurodegenerative diseases. This is the first report linking the trans-factor, SC35 to alternative splicing regulated by RA and the expression of the pro-survival protein PKCδVIII in neurons.

In summary, it is well established that Vitamin A and its metabolite RA directly affect transcription of genes. The inventors demonstrate herein that RA also regulates alternative splicing of genes. Previous studies demonstrated that RA reverses aging-related cognitive effects but no molecular mechanisms have been proposed to explain this. Further, understanding RA-mediated mechanisms of 5′ splice site selection and generation of PKCδ alternatively spliced variants will aid in the design of therapeutic interventions which will switch the splicing between the two isoforms. The inventors previously showed that using antisense oligonucleotides to mask 5′ splice sites promotes the selection of specific PKCδ splice variants. In the aging brain, switching the isoform expression to PKCδVIII by RA could shield the cells from neuronal death. This may influence the outcome of RA treatment to improve cognition and promote neurogenesis and provide a significant advantage without retinoid toxicity complications.

The inventors also found that human PKCδVIII expression is increased in neuronal cancer and decreased in Alzheimer's disease. The data shows that PKCδVIII promotes neuronal survival and increases neurogenesis via Bcl2 and Bcl-xL. In addition, the trans-factor SC35 was found to be crucial in mediating the effects of RA on alternative splicing of PKCδVIII mRNA in neurons. The data described herein indicate that PKCδVIII can be used as a biomarker for neurological diseases such as cancers and Alzheimer's disease and as a tool for monitoring and evaluating treatment.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of predicting neurodegenerative disease comprising:
obtaining the expression levels of PKCδVIII in a test tissue; and
comparing the expression levels of PKCδVIII to a predetermined control expression level;
wherein a decrease in expression levels indicates neurodegenerative disease.

2. The method of claim 1, wherein the neurodegenerative disease is selected from the group consisting of Alzheimer's disease, Parkinson's disease, Huntington's disease, dementia, amyotrophic lateral sclerosis, and multiple sclerosis.

3. A method of predicting neuronal metastases comprising:
obtaining the expression levels of PKCδVIII in a test tissue; and
comparing the expression levels of PKCδVIII to a predetermined control expression level;
wherein an increase in expression levels indicates neuronal metastases.

4. The method of claim 3, wherein the neuronal metastases are selected from the group consisting of gliomas and neuroblastomas.

5. A method of modulating expression of PKCδ isozymes in cells comprising administering an effective amount of a compound that affects the splicing enhancer SC35.

6. The method of claim 5, wherein the compound increases levels of splicing enhancer SC35.

7. The method of claim 5, wherein the compound administered is retinoic acid.

8. The method of claim 7, wherein the retinoic acid is all trans retinoic acid.

9. The method of claim 7, wherein the retinoic acid is administered to the cells for about 24 hours.

10. The method of claim 7, wherein the amount of retinoic acid administered to the cells is about 10 μM.

11. The method of claim 5, wherein the compound increases expression of PKCδVIII.

12. The method of claim 11, wherein the compound administered is retinoic acid.

13. The method of claim 11, wherein the retinoic acid is all trans retinoic acid.

14. The method of claim 11, wherein the retinoic acid is administered to the cells for about 24 hours.

15. The method of claim 11, wherein the amount of retinoic acid administered to the cells is about 10 μM.

16. A method of modulating neuronal cell survival in a subject comprising modulating levels of PKCδ isozymes.

17. The method of claim 16, wherein neuronal cell survival is increased by increasing levels of PKCδVIII.

18. The method of claim 17, wherein the level of PKCδVIII is increased by administering an effective amount of retinoic acid to the cells.

19. The method of claim 17, wherein the level of PKCδVIII is increased by increasing amounts of splicing enhancer SC35 in the cell.

20. A method of modulating apoptosis in cells comprising modulating levels of PKCδ isozymes.

21. The method of claim 20 wherein apoptosis is decreased by increasing levels of PKCδVIII.

22. The method of claim 21 wherein the level of PKCδVIII is increased by administering an effective amount of retinoic acid to the cells.

23. The method of claim 21 wherein the level of PKCδVIII is increased by increasing amounts of splicing enhancer SC35 in the cell.


SEQUENCE LISTING (22 sequences)

SEQ ID NO: 1 - protein, 25 aa, artificial sequence
  amino acids 329 to 351 of PKC delta VIII
HISGEAGSIAPLRFLFPLRPKKGDC

SEQ ID NO: 2 - DNA, 19 bp, artificial sequence
  PKC delta I sense primer
gccaacctctgcggcatca

SEQ ID NO: 3 - DNA, 26 bp, artificial sequence
  PKC delta I antisense primer
cgtaggtcccactgttgtcttgcatg

SEQ ID NO: 4 - DNA, 19 bp, artificial sequence
  PKC delta VIII sense primer
gccaacctctgcggcatca

SEQ ID NO: 5 - DNA, 26 bp, artificial sequence
  PKC delta VIII antisense primer
cgtaggtcccactgttgtcctgtctc

SEQ ID NO: 6 - DNA, 21 bp, artificial sequence
  GADPH sense primer
cttcattgacctcaactacat

SEQ ID NO: 7 - DNA, 21 bp, artificial sequence
  GADPH antisense primer
tgtcatggatgaccttggcca

SEQ ID NO: 8 - DNA, 22 bp, artificial sequence
  human PKC delta sense primer
cactatattccagaaagaacgc

SEQ ID NO: 9 - DNA, 17 bp, artificial sequence
  human PKC delta VIII antisense primer
ccctcccagatcttgcc

SEQ ID NO: 10 - DNA, 17 bp, artificial sequence
  PKC delta specific antisense primer
ccctcccagatcttgcc

SEQ ID NO: 11 - DNA, 20 bp, artificial sequence
  SD-SA on pSPL3 sense primer
tctcagtcacctggacaacc

SEQ ID NO: 12 - DNA, 21 bp, artificial sequence
  SD-SA pSPL3 antisense primer
ccacaccagccaccaccttct

SEQ ID NO: 13 - DNA, 20 bp, artificial sequence
  SC35 sense primer
tccaagtccaagtcctcctc

SEQ ID NO: 14 - DNA, 20 bp, artificial sequence
  SC35 antisense primer
actgctccctcttcttctgg

SEQ ID NO: 15 - DNA, 29 bp, artificial sequence
  forward primer for pSPL3-PKC delta minigene
ccttgatcatgggagttctgataatggtc

SEQ ID NO: 16 - DNA, 28 bp, artificial sequence
  reverse primer for pSPL3-PKC delta minigene
cctactagtatcgggtctcagtctacac

SEQ ID NO: 17 - DNA, 8 bp, artificial sequence
  SC35 cis element
ggccaaag

SEQ ID NO: 18 - DNA, 9 bp, artificial sequence
  SC35 mutated cis element
tagcccata

SEQ ID NO: 19 - DNA, 27 bp, artificial sequence
  forward primer for splicing template
ccttctcgagctgggctgggagttctg

SEQ ID NO: 20 - DNA, 26 bp, artificial sequence
  reverse primer for splicing template
cccacctcagccacgcggccgcctaa

SEQ ID NO: 21 - DNA, 24 bp, artificial sequence
  first product reverse primer for splicing template
cggtggttccttccccggtacctg

SEQ ID NO: 22 - DNA, 24 bp, artificial sequence
  second product forward primer for splicing template
tcggtaccgggcagacaacagtgg